(19) United States
(12) Patent Application Publication
(10) Pub. No.: US 2022/0081724 A1
(43) Pub. Date: Mar. 17, 2022

(54) METHODS OF DETECTING AND TREATING SUBJECTS WITH CHECKPOINT INHIBITOR-RESPONSIVE CANCER

(71) Applicant: Strata Oncology, Inc., Ann Arbor, MI (US)

(72) Inventors: Daniel Reed Rhodes, Chelsea, MI (US); Scott Arthur Tomlins, Ann Arbor, MI (US); David Bryan Johnson, Chelsea, MI (US)

(21) Appl. No.: 17/416,966

(22) PCT Filed: Dec. 19, 2019
(86) PCT No.: PCT/US2019/067673

§ 371 Date: Jun. 21, 2021

Related U.S. Application Data
(60) Provisional application No. 62/782,198, filed on Dec. 19, 2018.

(51) Int. Cl. C12Q 1/6886

(52) U.S. Cl. CPC C12Q 1/6886; C12Q 2600/156; C12Q 2600/158; C12Q 2600/106

(57) ABSTRACT

Disclosed herein are methods of detecting and treating checkpoint inhibitor responsive cancers comprising calculating, determining, or obtaining PD-L1 expression, CD8A expression, and tumor content from a cancer specimen.

### RELATED APPLICATION(S)

This application claims the benefit of U.S. Provisional Application No. 62/782,198, filed on Dec. 19, 2018, the contents of which are hereby incorporated by reference in their entirety.

### BACKGROUND OF THE INVENTION

Checkpoint inhibitors (i.e., PD 1/PD-L1 inhibition) have been widely used in cancer treatment and have impressive survival benefits. However, activation of the immune system via checkpoint inhibitors can cause a number of adverse events that can cause morbidity or mortality. Common serious adverse events include colitis, hepatitis, adrenocorticotropic hormone insufficiency, hypothyroidism, type 1 diabetes, acute kidney injury and myocarditis. Thus, it has become desirable to identify subjects with cancers responsive to checkpoint inhibition prior to commencing checkpoint inhibition therapy.

Several biomarkers have been explored to evaluate those that are predictive of response for PD 1/PD-L1 inhibition. These include PD-L1 expression (by immunohistochemistry), tumor infiltrating lymphocytes (such as effector CD8-positive T cells), T-cell receptor clonality, TMB, MSI status, peripheral blood markers, immune gene signatures, and multiplex immunohistochemistry (Gibney et al, 2016). The most well-studied biomarker is PD L1 expression, which is approved as a companion or complementary diagnostic for multiple checkpoint inhibitors. While PD-L1 expression enriches for response in some indications, it is not a perfect biomarker, with many biomarker-positive patients exhibiting little treatment response and biomarker-negative patients exhibiting substantial response (Larkin et al, 2015; Borghaei et al, 2015; Brahmer et al, 2015; Garon et al, 2015; Mahoney et al, 2014). Likewise, multiple antibodies, staining protocols, and evaluation methodologies are utilized (eg, some approaches only consider PD-L1 expression on tumor cells, while others consider both tumor and immune cell expression). Similarly, the use of biomarkers beyond PD-L1 to identify patient subgroups who will respond to checkpoint inhibitors or who will have an increased risk of off-target effects (such as development of an autoimmune disease) has not yet led to a clear patient stratification biomarker (Gibney et al, 2016; Topalian et al, 2016).

Recently, pembrolizumab was approved for patients with MSI-H or deoxyribonucleic acid (DNA) mismatch repair defects, irrespective of tumor type (Le et al, 2017). The registration-enabling clinical trial was conducted as an investigator-initiated trial and enrolled biomarker-positive patients across a range of tumor types. Fifty-four percent (54%; 95% confidence interval 39% to 69%) of patients had an objective response at 20 weeks and 1-year overall survival estimate of 76% (Le et al, 2017). MSI-H is more common in colorectal (17%) and endometrial cancer (28%) but is relatively rare in other tumor types, ranging from 0.2% to 5.4% across 16 cancer types (Ashktorab et al, 2016; Cortes-Ciriano, et al, 2017). MSI-H is thought to confer sensitivity to checkpoint inhibitors due to the substantially increased tumor mutational burden in MSI-H tumors, leading to an abundance of neoantigens and a robust tumor immune response, which is abrogated through immune checkpoint pathways. Although representing the first tumor-agnostic biomarker-based drug approval, MSI-H tumors are speculated to represent only a fraction of tumor types outside of approved indications that are likely to respond to checkpoint therapy. Thus, there remains a need for biomarker assays to detect tumors responsive to checkpoint inhibition.

### SUMMARY OF THE INVENTION

Some aspects of the present disclosure are related to a method of treatment comprising calculating, determining, or obtaining PD-L1 expression, CD8A expression, and tumor content in a tumor specimen from a subject to identify the subject as having a checkpoint inhibitor responsive cancer; and administering a checkpoint inhibitor therapy to the identified subject. In some embodiments, one or more of the following are also calculated, determined, or obtained for the tumor specimen: the presence of chimeric transcripts indicative of gene fusion, cDNA sequence data from cDNA converted from mRNA, DNA sequence data, tumor mutation burden (TMB)-associated data, and microsatellite instability (MSI)-associated data. In some embodiments, tumor mutation burden (TMB)-associated data is also calculated, determined, or obtained for the tumor specimen. In some embodiments, the tumor specimen is a formalin-fixed paraffin-embedded (FFPE) tumor specimen. In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

In some embodiments, PD-L1 expression is calculated using PCR and next-generation sequencing or is determined from PCR and next-generation sequencing data. In some embodiments, PD-L1 expression is calculated by normalizing read data to one or more housekeeping genes including one or more of: LRP1, MRPL13, TBP, HMBS, ITGB7, MYC, CIAO1, CTCF, EIF2B1, GGNBP2, SLC4A1AP and/or other suitable housekeeping genes (and/or any suitable genes). In some embodiments, the housekeeping genes comprise or consist of EIF2B1, HMBS, CIAO1. In some embodiments, PD-L1 expression data is obtained from another party.

In some embodiments, the subject is identified as having a checkpoint inhibitor responsive cancer when the PD-L1 expression is calculated as or determined to be high. In some embodiments, high PD-L1 expression is calculated or determined to be at least the 70^{th} (e.g., the 73.3) percentile based upon a population of tumor profiles (i.e., at the 70^{th} or higher percentile in a ranking of tumor profiles for PD-L1 expression). In some embodiments of the methods disclosed herein, the population of tumor profiles includes at least 5, at least 10, at least 15, at least 20, at least 30, at least 50, at least 100, at least 200, at least 500, or more profiles of individual tumors. In some embodiments, high PD-L1 expression equals 2,000 normalized reads per million or more. In some embodiments, the calculated PD-L1 expression is confirmed or combined with a secondary measurement of PD-L1 expression using a second amplicon, and wherein the secondary measurement's percentile value is 80% or more of the calculated PD-L1 percentile value.

In some embodiments, CD8A expression is calculated using PCR and next-generation sequencing. In some embodiments, the subject is identified as having a checkpoint inhibitor responsive cancer when the CD8A expression is calculated as high. In some embodiments, high CD8A expression equals 10,000 normalized reads per million or more. In some embodiments, the calculated CD8A expression is confirmed or combined with a secondary measurement of GZMA expression using a second amplicon, and wherein the secondary measurement's percentile value is 80% or more of the calculated CD8A expression value.

In some embodiments, the tumor specimen has a tumor content of 40% or more. In some embodiments, the subject is identified as having a checkpoint inhibitor responsive cancer when the PD-L1 expression is calculated as high, the CD8A expression is calculated as high, and the tumor content of the tumor specimen is 40% or more. In some embodiments, the subject is identified as having a checkpoint inhibitor responsive cancer when the PD-L1 expression of the tumor specimen is calculated as high, the CD8A expression of the tumor specimen is calculated as high, and the tumor content of the tumor specimen is 40% or more, or wherein the subject is identified as having a checkpoint inhibitor responsive cancer when the TMB of the tumor specimen is 15 or more mutations per megabase (Mb).

In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is an anti-PD-1 antibody or an anti-PD-L1 antibody. In some embodiments, the checkpoint inhibitor is an antibody that inhibits two or more of the checkpoint proteins selected from the group of PD-1, CTLA-4, PD-L1 and PD-L2. In some embodiments, the checkpoint inhibitor is nivolumab, pembrolizumab, atezolizumab, durvalumab, pidilizumab, PDR001, BMS-936559, avelumab, SHR-1210 or AB122.

Some aspects of the present disclosure are related to a method of identifying whether a subject has a checkpoint inhibitor responsive cancer comprising calculating PD-L1 expression, CD8A expression, and tumor content in a tumor specimen from a subject to identify whether the subject has a checkpoint inhibitor responsive cancer. In some embodiments, one or more of the following are also calculated for the tumor specimen: the presence of chimeric transcripts indicative of gene fusion, cDNA sequence data from cDNA converted from mRNA, DNA sequence data, tumor mutation burden (TMB)-associated data, and microsatellite instability (MSI)-associated data. In some embodiments, tumor mutation burden (TMB)-associated data is also calculated for the tumor specimen. In some embodiments, the tumor specimen is a formalin-fixed paraffin-embedded (FFPE) tumor specimen.

In some embodiments, the tumor specimen is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

In some embodiments, PD-L1 expression is calculated using PCR and next-generation sequencing. In some embodiments, the subject is identified as having a checkpoint inhibitor responsive cancer when the PD-L1 expression is calculated as high. In some embodiments, high PD-L1 expression is calculated or determined to be at least the 73^{th} (e.g., 73.3) percentile of PD-L1 expression across a population of tumor profiles. In some embodiments, high PD-L1 expression equals 2,000 normalized reads per million or more. In some embodiments, the calculated PD-L1 expression is confirmed or combined with a secondary measurement of PD-L1 expression using a second amplicon. In some embodiments the secondary measurement's percentile value is 80% or more of the calculated PD-L1 percentile value.

In some embodiments, CD8A expression is calculated using PCR and next-generation sequencing or is determined from PCR and next-generation sequencing data. In some embodiments, CD8A expression is calculated by normalizing read data to one or more housekeeping genes including one or more of: LRP1, MRPL13, TBP, HMBS, ITGB7, MYC, CIAO1, CTCF, EIF2B1, GGNBP2, SLC4A1AP and/or other suitable housekeeping genes (and/or any suitable genes). In some embodiments, the housekeeping genes comprise or consist of EIF2B1, HMBS, CIAO1. In some embodiments, CD8A expression data is obtained from another party.

In some embodiments, the subject is identified as having a checkpoint inhibitor responsive cancer when the CD8A expression is calculated as or determined to be high. In some embodiments, high CD8A expression is calculated or determined to be at least the 67^{th} (e.g., 67.6) percentile of CD8A expression across a population of tumor profiles. In some embodiments, high CD8A expression equals 10,000 normalized reads per million or more. In some embodiments, the calculated CD8A expression is confirmed or combined with a secondary measurement of a CD8A-related transcripts' expression, including GZMA, GZMB, GZMK, PRF1, IFNG or CD8B. In some embodiments, CD8A expression is confirmed or combined with a secondary measurement of GZMA expression using a second amplicon, and wherein the secondary measurement's percentile value is 80% or more of the calculated CD8A percentile value.

In some embodiments, the tumor specimen has a tumor content of 40% or more. In some embodiments, the tumor specimen has a tumor content of 20% or more.

In some embodiments, the subject is identified as having a checkpoint inhibitor responsive cancer when the PD-L1 expression is calculated as high, the CD8A expression is calculated as high, and the tumor content of the tumor specimen is 40% or more. In some embodiments, the subject is identified as having a checkpoint inhibitor responsive cancer when the PD-L1 expression of the tumor specimen is calculated as high, the CD8A expression of the tumor specimen is calculated as high, and the tumor content of the tumor specimen is 40% or more, or wherein the subject is identified as having a checkpoint inhibitor responsive cancer when the TMB of the tumor specimen is 15 or more mutations per megabase (Mb). In some embodiments, the subject is identified as having a checkpoint inhibitor responsive cancer when the TMB of the tumor specimen is 15 or more mutations per megabase (Mb) and the tumor content is at least 20%.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides a flow representation of variations of an embodiment of a method 100.

FIG. 2 provides a flow representation of variations of an embodiment of a method 100.

FIG. 3 provides a flow representation of variations of an embodiment of a method 100.

FIG. 4 is a graph showing the results of the screen in Example 1. Tumors responsive to checkpoint inhibition are shown in orange. Dotted lines indicate CD8A high and PD-L1 high expression as defined in Example 1.

FIG. 5 is a graph of TMB testing shown in Example 1. The dotted line indicates 18 mutations per megabyte. “R” signifies tumors responsive to checkpoint inhibition.

FIG. 6 is a graph showing concordance between the PD-L1 primary amplicon and secondary amplicon.

FIG. 7 is a graph showing concordance between CD8A primary amplicon and GZMA amplicon.

FIG. 8 are graphs showing percentile ratios between PD-L1 amplicons (left side) or GZMA and CD8A (right side).

FIG. 9 are graphs comparing the results of the screens for CD8A-High/PD-L1—high tumors in Example 1 (left side) and Example 2 (right side).

FIG. 10 is a graph showing the results of a screen by the method shown in Example 2.

FIG. 11 shows the results of a TMB screen. Top dotted line indicates TMB-H (15 mutations/megabase).

FIG. 12 provides TMB-H and PD-L1+CD8A high subjects (left graphs) and the response of these two combined groups to anti-PD-1 therapy (right graph).

FIG. 13 is a Venn diagram of TMB, MSI, and SIS (PD-L1/CD8A high) patient populations showing overlap between these groups.

FIG. 14 shows an example scenario for the method of Example 2 wherein the tumor is PD-L1 High/CD8A High/TC High (SIS positive).

FIG. 15 shows an example scenario for the method of Example 2 wherein the tumor is PD-L1 Low/CD8A Low/TC High (SIS negative).

FIG. 16 shows an example scenario for the method of Example 2 wherein the tumor is PD-L1 High/CD8A High/TC Low (SIS negative).

FIG. 17 shows an example scenario for the method of Example 2 wherein the tumor is PD-L1 High/CD8A Low/TC High (SIS negative).

FIG. 18 shows an example scenario for the method of Example 2 wherein the tumor is PD-L1 Low/CD8A High/TC High (SIS negative).

### DETAILED DESCRIPTION OF THE INVENTION

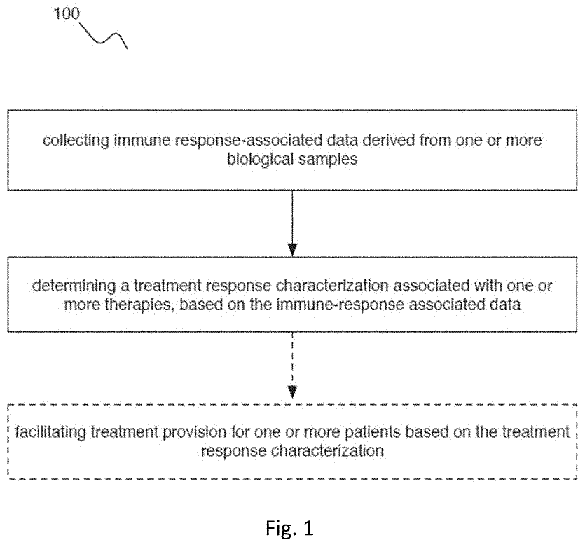

Some aspects of the present disclosure are directed to a method (e.g., a method 100 of FIGS. 1-3) for identifying a subject (sometimes referred herein as a patient) who will and/or are more likely to respond positively to PD-1/PD-L1 inhibitor therapy and/or suitable immune checkpoint therapies (i.e., a subject having a checkpoint inhibitor responsive cancer). In some embodiments, the subject has a tumor and the method comprises calculating, determining or obtaining data showing if the tumor will be or is more likely to be responsive to PD-1/PD-L1 inhibitor therapy and/or suitable immune checkpoint therapies (sometimes referred to herein as a “checkpoint inhibitor responsive cancer”). In some embodiments, the method further comprises administering PD-1/PD-L1 inhibitor therapy and/or suitable immune checkpoint therapy (sometimes referred to herein as a “checkpoint inhibitor”) to the identified subject or tumor. In some embodiments, a subject responsive to a checkpoint inhibitor does not have disease progression within 12 months of beginning a checkpoint inhibitor therapy.

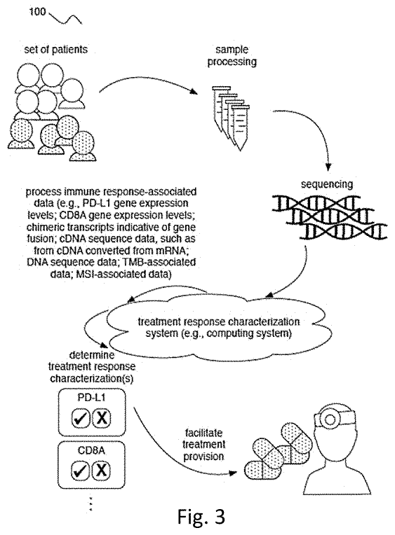

As shown in FIGS. 1 and 3, embodiments of a method 100 (e.g., for identifying patients who will and/or are more likely to respond positively to PD-1/PD-L1 inhibitor therapy and/or suitable immune checkpoint therapies; etc.) can include: collecting immune response-associated data (e.g., programmed death-ligand 1 (PD-L1) gene expression levels; Cluster of Differentiation 8a (CD8A) gene expression levels; chimeric transcripts indicative of gene fusion; cDNA sequence data, such as from cDNA converted from mRNA; DNA sequence data; tumor mutation burden (TMB)-associated data; microsatellite instability (MSI)-associated data; etc.) derived from one or more biological samples (e.g., formalin-fixed paraffin-embedded (FFPE) tumor specimens; suitable tumor specimens; etc.); and determining a treatment response characterization associated with one or more therapies (e.g., responsiveness to immune checkpoint therapies such as PD-1/PD-L1 inhibitor therapy and/or other suitable therapies; etc.), based on the immune-response associated data. Additionally or alternatively, embodiments of the method 100 can include facilitating treatment provision for one or more patients based on the treatment response characterization; and/or can include any suitable processes.

In some embodiments, determining if the tumor will be or is more likely to be responsive to PD-1/PD-L1 inhibitor therapy and/or suitable immune checkpoint therapy comprises collecting or providing a tumor specimen from a subject. In some embodiments, the tumor specimen is a fresh tumor specimen or a formalin-fixed paraffin-embedded (FFPE) tumor specimen. However, the specimen preparation is not limited and may be any suitable preparation known in the art. In some embodiments, the methods do not include collecting or providing a tumor. Instead, data or a qualitative assessment (e.g., a determination that the tumor has high or low expression of a relevant marker or high or low tumor content) is provided. In some embodiments, the data or qualitative assessment is provided to a physician or other health professional and such person uses such data or assessment to determine whether or not to administer the PD-1/PD-L1 inhibitor therapy and/or suitable immune checkpoint therapy. The provided data or qualitative assessment can be calculated or determined by any of the methods disclosed herein.

The tumor may be from any cancer is not limited. As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid cancer.

In some embodiments, the cancer is not a blood-borne or hematopoietic cancer. In some embodiments, the cancer is not an MSI-H cancer. In some embodiments, the cancer is not 1, 2, 3, 4, 5, 6 or all 7 of melanoma, lung cancer, kidney cancer, bladder cancer, head and neck cancer, and Hodgkin's lymphoma. In some embodiments, the cancer is adrenal cancer, biliary cancer, bladder cancer, brain cancer, breast cancer, cervical cancer, colon cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

In some embodiments, determining or calculating if the tumor will be or is more likely to be responsive to PD-1/PD-L1 inhibitor therapy and/or suitable immune checkpoint therapy comprises calculating, collecting or determining immune-response associated data derived from the tumor. In some embodiments, the methods disclosed herein comprise obtaining immune-response associated data (quantitative or qualitative) derived from the tumor from another party and determining if the tumor will be or is more likely to be responsive to PD-1/PD-L1 inhibitor therapy and/or suitable immune checkpoint therapy.

In some embodiments, the immune-response associated data comprises one or more of programmed death-ligand 1 (PD-L1) gene expression levels; Cluster of Differentiation 8a (CD8A) gene expression levels; chimeric transcripts indicative of gene fusion; cDNA sequence data, such as from cDNA converted from mRNA; DNA sequence data; tumor mutation burden (TMB)-associated data; microsatellite instability (MSI)-associated data. In some embodiments, at least two, at least three, at least four, at least five or more immune-response associated data types (e.g., programmed death-ligand 1 (PD-L1) gene expression levels; Cluster of Differentiation 8a (CD8A) gene expression levels; chimeric transcripts indicative of gene fusion; cDNA sequence data, such as from cDNA converted from mRNA; DNA sequence data; tumor mutation burden (TMB)-associated data; microsatellite instability (MSI)-associated data) are calculated, collected, or determined. In some embodiments, immune-response associated data is collected or determined via NGS and/or multiplexed PCR. In some embodiments, immune-response associated data is obtained from NGS and/or multiplexed PCR performed by another party.

In some embodiments, programmed death-ligand 1 (PD-L1) gene expression levels and Cluster of Differentiation 8a (CD8A) gene expression levels are determined, calculated or obtained. In some embodiments, programmed death-ligand 1 (PD-L1) gene expression levels, Cluster of Differentiation 8a (CD8A) gene expression levels, and MSI associated data are determined, calculated or obtained. In some embodiments, programmed death-ligand 1 (PD-L1) gene expression levels, Cluster of Differentiation 8a (CD8A) gene expression levels, and TMB associated data are determined, calculated or obtained. In some embodiments, programmed death-ligand 1 (PD-L1) gene expression levels, Cluster of Differentiation 8a (CD8A) gene expression levels, TMB associated data, and MSI associated data are determined, calculated or obtained.

In some embodiments, PD-L1 expression is determined or calculated via NGS of gene expression transcripts using multiplex PCR (amplicon). In some embodiments, PD-L1 expression is obtained from NGS of gene expression transcripts using multiplex PCR (amplicon) data. In some embodiments, PD-L1 expression is validated, confirmed, or combined using multiplex PCR and a second amplicon. In some embodiments, validation or confirmation of PD-L1 requires that the second amplicon's percentile value is 70%, 75%, 80%, 85% or more of the calculated PD-L1 percentile value. In some embodiments, validation or confirmation of PD-L1 requires that the second amplicon's percentile value is 80% or more of the calculated PD-L1 percentile value.

In some embodiments, CD8A expression is determined or calculated via NGS of gene expression transcripts using multiplex PCR (amplicon). In some embodiments, CD8A expression is obtained from NGS of gene expression transcripts using multiplex PCR (amplicon) data. In some embodiments, CD8A expression is validated, confirmed, or combined using multiplex PCR (amplicon) to measure GZMA, GZMB, GZMK, PRF1, IFNG or CD8B expression. In some embodiments, CD8A expression is validated, confirmed, or combined using multiplex PCR (amplicon) to measure GZMA expression. CD8A and GZMA are both part of the interferon-y gene signature. In some embodiments, validation, confirmation or combination of CD8A requires that the second amplicon (e.g., GZMA) measurement's percentile value is 80% or more of the calculated CD8A percentile value.

In some embodiments, TMB is determined or calculated by NGS of tumor DNA. In some embodiments, TMB is obtained from another party.

In some embodiments the methods further comprise determining, calculating or obtaining tumor content of the tumor specimen. Methods of determining or calculating tumor content are not limited and may be any suitable method known in the art. In some embodiments, tumor content is determined by histopathology by a pathologist. In some embodiments, tumor content is determined by assessing molecular tumor content from sequence data obtained from the specimen. In some embodiments, molecular tumor content is determined by triangulating on three independent inputs: (1) Somatic mutation variant allele frequency (VAF) (e.g., for homozygous mutations in tumor suppressors, VAF approximates tumor content; for heterozygous oncogene mutations at neutral copy number, VAF*2 approximates tumor content). (2) Step function from segmented copy number profile (i.e., steps should equal 1.0 copies for 100% tumor content in diploid tumors, 0.5 for 50% tumor content, etc.). (3) Germline VAF within regions of copy number change (e.g., heterozygous germline variants will have ˜50% VAF at positions with 2 copies; for positions with 1 copy loss and 100% tumor content, germline variants will have ˜100% or ˜0% VAF; etc.).

In some embodiments, tumor specimens must have about 20% tumor content or more in order to determine if the tumor will be or is more likely to be responsive to PD-1/PD-L1 inhibitor therapy and/or suitable immune checkpoint therapy. In some embodiments, tumor specimens must have about 25% tumor content or more in order to determine if the tumor will be or is more likely to be responsive to PD-1/PD-L1 inhibitor therapy and/or suitable immune checkpoint therapy. In some embodiments, tumor specimens must have about 30% tumor content or more in order to determine if the tumor will be or is more likely to be responsive to PD-1/PD-L1 inhibitor therapy and/or suitable immune checkpoint therapy. In some embodiments, tumor specimens must have about 35% tumor content or more in order to determine if the tumor will be or is more likely to be responsive to PD-1/PD-L1 inhibitor therapy and/or suitable immune checkpoint therapy. In some embodiments, tumor specimens must have about 40% tumor content or more in order to determine if the tumor will be or is more likely to be responsive to PD-1/PD-L1 inhibitor therapy and/or suitable immune checkpoint therapy. In some embodiments, tumor specimens must have about 45% tumor content or more in order to determine if the tumor will be or is more likely to be responsive to PD-1/PD-L1 inhibitor therapy and/or suitable immune checkpoint therapy. In some embodiments, tumor specimens must have about 50% tumor content or more in order to determine if the tumor will be or is more likely to be responsive to PD-1/PD-L1 inhibitor therapy and/or suitable immune checkpoint therapy. In some embodiments, tumor specimens must have about 55% tumor content or more in order to determine if the tumor will be or is more likely to be responsive to PD-1/PD-L1 inhibitor therapy and/or suitable immune checkpoint therapy. In some embodiments, tumor specimens must have about 60% tumor content or more in order to determine if the tumor will be or is more likely to be responsive to PD-1/PD-L1 inhibitor therapy and/or suitable immune checkpoint therapy.

In some embodiments, a cancer or subject will be or is more likely to be responsive to PD-1/PD-L1 inhibitor therapy and/or suitable immune checkpoint therapy if the tumor specimen has high PD-L1 expression. In some embodiments, high PD-L1 expression is calculated or determined to be at least the 68, 69, 70^{th}, 71^{st}, 72^{nd}, 73^{rd}, 74^{th}, 75^{th}, 76^{th}, 77^{th}, 78^{th}, 79^{th}, or 80^{th} percentile based upon a population of tumor profiles. In some embodiments, high PD-L1 expression is calculated or determined to be at least the 73.3 percentile based upon a population of tumor profiles. In some embodiments of the methods disclosed herein, the population of tumor profiles includes at least 5, at least 10, at least 15, at least 20, at least 30, at least 50, at least 100, at least 200, at least 500, or more profiles of individual tumors. In some embodiments, high PD-L1 expression is defined as equal to or above the point on each biomarker's receiver-operating characteristic (ROC) curve that maximized Youden's J statistic. In some embodiments, high PD-L1 expression is defined as about 14K (i.e., 14,000) normalized reads per million [nRPM] or more.

In some embodiments, the subject is identified as having a checkpoint inhibitor responsive cancer when the CD8A expression is calculated as or determined to be high. In some embodiments, high CD8A expression is calculated or determined to be at least the 60^{th}, 61^{st}, 62^{nd}, 63^{rd}, 64^{th}, 65^{th}, 66^{th}, 67^{th}, 68^{th}, 69^{th}, or 70^{th} percentile of CD8A across a population of tumor profiles. In some embodiments, high CD8A expression is calculated or determined to be at least the e.g., 67.6 percentile of CD8A across a population of tumor profiles. In some embodiments, high CD8A expression is defined as equal to or above the point on each biomarker's receiver-operating characteristic (ROC) curve that maximized Youden's J statistic. In some embodiments, high CD8A expression is defined as about 69K normalized reads per million [nRPM] or more.

In some embodiments, a cancer or subject will be or is more likely to be responsive to PD-1/PD-L1 inhibitor therapy and/or suitable immune checkpoint therapy if the tumor specimen has high PD-L1 expression, high CD8A expression, and a tumor content (e.g., molecular tumor content) of at least 20%, at least 30%, at least 40%, at least 50%, at least 60% or more. In some embodiments, a cancer or subject will be or is more likely to be responsive to PD-1/PD-L1 inhibitor therapy and/or suitable immune checkpoint therapy if the tumor specimen has high PD-L1 expression, high CD8A expression, and a tumor content (e.g., molecular tumor content) of at least 50% or more. In some embodiments, a cancer or subject will be or is more likely to be responsive to PD-1/PD-L1 inhibitor therapy and/or suitable immune checkpoint therapy if the tumor specimen has PD-L1 expression of 14K nRPM or more (i.e., 73.3 percentile or more), CD8A expression of 69K nRPM or more (i.e., 67.6 percentile or more), and a tumor content (e.g., molecular tumor content) of 50% or more.

In some embodiments, a cancer or subject will be or is more likely to be responsive to PD-1/PD-L1 inhibitor therapy and/or suitable immune checkpoint therapy if the tumor specimen has high PD-L1 expression in a primary measurement with a secondary PD-L1 measurement (e.g., a second amplicon) percentile value of 80% or more of the primary measurement, high CD8A expression in a primary measurement with a secondary GZMA, GZMB, GZMK, PRF1, IFNG or CD8B measurement (e.g., a second amplicon) percentile value of 80% or more of the primary measurement, and a tumor content (e.g., molecular tumor content) of 40% or more. In some embodiments, a cancer or subject will be or is more likely to be responsive to PD-1/PD-L1 inhibitor therapy and/or suitable immune checkpoint therapy if the tumor specimen has high PD-L1 expression in a primary measurement with a secondary PD-L1 measurement (e.g., a second amplicon) percentile value of 80% or more of the primary measurement, high CD8A expression in a primary measurement with a secondary GZMA measurement (e.g., a second amplicon) percentile value of 80% or more of the primary measurement, and a tumor content (e.g., molecular tumor content) of 40% or more. In some embodiments, a cancer or subject will be or is more likely to be responsive to PD-1/PD-L1 inhibitor therapy and/or suitable immune checkpoint therapy if the tumor specimen has PD-L1 expression of 2K nRPM or more with a secondary PD-L1 measurement (e.g., a second amplicon) percentile value of 80% or more of the primary measurement, CD8A expression of 10K nRPM or more with a secondary GZMA, GZMB, GZMK, PRF1, IFNG or CD8B measurement (e.g., a second amplicon) percentile value of 80% or more of the primary measurement, and a tumor content (e.g., molecular tumor content) of 40% or more. In some embodiments, a cancer or subject will be or is more likely to be responsive to PD-1/PD-L1 inhibitor therapy and/or suitable immune checkpoint therapy if the tumor specimen has PD-L1 expression of 2K nRPM or more with a secondary PD-L1 measurement (e.g., a second amplicon) percentile value of 80% or more of the primary measurement, CD8A expression of 10K nRPM or more with a secondary GZMA measurement (e.g., a second amplicon) percentile value of 80% or more of the primary measurement, and a tumor content (e.g., molecular tumor content) of 40% or more.

In some embodiments, methods disclosed herein of detecting a tumor responsive to checkpoint inhibition by detecting a PD-L1 high and CD8A high signature has an adjusted positive predictive value (PPV) of at least 40%, 41%, 42%, 43%, 44%, 45% or more, assuming a pan-cancer unselected checkpoint inhibitor response rate of 10%. In some embodiments, methods disclosed herein of detecting a tumor responsive to checkpoint inhibition by detecting a PD-L1 high and CD8A high signature has an adjusted positive predictive value (PPV) of at least 44% or 44.9% or more, assuming a pan-cancer unselected checkpoint inhibitor response rate of 10%. In some embodiments, methods disclosed herein of detecting a tumor responsive to checkpoint inhibition by detecting a PD-L1 high and CD8A high signature has a specificity of at least 90%, 91%, 92%, 93%, 94%, 95%, 96%, 97%, 98%, 99% or more. In some embodiments, methods disclosed herein of detecting a tumor responsive to checkpoint inhibition by detecting a PD-L1 high and CD8A high signature has a specificity of at least 95% or 95.5%.

In some embodiments, methods disclosed herein of detecting a tumor responsive to checkpoint inhibition by detecting a PD-L1 high and CD8A high signature, or a TMB high signature, has an adjusted positive predictive value (PPV) of at least 40%, 41%, 42%, 43%, 44%, 45% or more, assuming a pan-cancer unselected checkpoint inhibitor response rate of 10%. In some embodiments, methods disclosed herein of detecting a tumor responsive to checkpoint inhibition by detecting a PD-L1 high and CD8A high signature, or a TMB high signature, has an adjusted positive predictive value (PPV) of at least 44% or more, assuming a pan-cancer unselected checkpoint inhibitor response rate of 10%. In some embodiments, methods disclosed herein of detecting a tumor responsive to checkpoint inhibition by detecting a PD-L1 high and CD8A high signature, or a TMB high signature, can detect at least about 60%, 61%, 62%, 63%, 64%, 65%, 66%, 67%, 68%, 69%, 70% or more of checkpoint inhibitor responsive (e.g., PD-1/PD-L1 responsive) cancers. In some embodiments, methods disclosed herein of detecting a tumor responsive to checkpoint inhibition by detecting a PD-L1 high and CD8A high signature, or a TMB high signature, can detect at least about 66% or more of checkpoint inhibitor responsive (e.g., PD-1/PD-L1 responsive) cancers.

In some embodiments, a cancer or subject will be or is more likely to be responsive to PD-1/PD-L1 inhibitor therapy and/or suitable immune checkpoint therapy if the tumor specimen has high PD-L1 expression, high CD8A expression and a tumor content of 40% or more, or if the tumor specimen is TMB high (TMB-H). In some embodiments, TMB-H is 15 or more mutations per megabase (Mb). In some embodiments, TMB-H is 10, 11, 12, 13, 14, 15, 16, 17, 18, 19, 20 or more mutations per Mb. In some embodiments, the tumor specimen has a tumor content of at least 20%.

Methods of detecting mutations (e.g., TMB) are not limited. In some embodiments, mutations are detected, calculated or obtained via NGS. In some embodiments, TMB includes non-coding (at highly characterized genomic loci) and coding, synonymous and non-synonymous, single and multi-nucleotide (two bases) variants present at >10% variant allele frequency (VAF). In some embodiments, mutations per megabase (Mb) estimates and associated 90% confidence interval are calculated via the total number of positions with sufficient depth of coverage necessary for definitive assessment (maximum possible 1.7 Mb).

In some embodiments, the checkpoint inhibitor administered is an antibody against at least one checkpoint protein, e.g., PD-1, CTLA-4, PD-L1 or PD-L2. In some embodiments, the checkpoint inhibitor administered is an antibody that is effective against two or more of the checkpoint proteins selected from the group of PD-1, CTLA-4, PD-L1 and PD-L2. In some embodiments, the checkpoint inhibitor administered is a small molecule, non-protein compound that inhibits at least one checkpoint protein. In one embodiment, the checkpoint inhibitor is a small molecule, non-protein compound that inhibits a checkpoint protein selected from the group consisting of PD-1, CTLA-4, PD-L1 and PD-L2. In some embodiments, the checkpoint inhibitor administered is nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), atezolizumab (Tecentriq®, Genentech/Roche, South San Francisco Calif.), durvalumab (MED14736, Medimmune/AstraZeneca), pidilizumab (CT-011, CureTech), PDR001 (Novartis), BMS-936559 (MDX1105, BristolMyers Squibb), avelumab (MSB0010718C, Merck Serono/Pfizer), or SHR-1210 (Incyte). Additional antibody PD1 pathway inhibitors for use in the methods described herein include those described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, and U.S. Pat. No. 7,943,743 (Medarex, Inc) issued May 17, 2011.

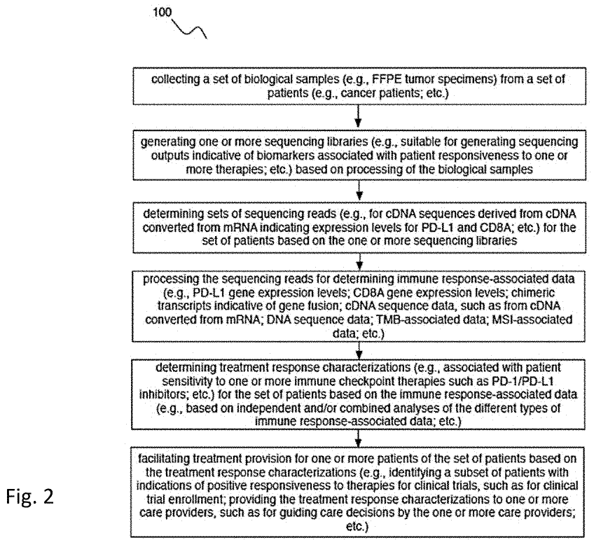

In a specific example, as shown in FIG. 2, the methods of the claimed invention (e.g., method 100) can include one or more of: collecting a set of biological samples (e.g., FFPE tumor specimens) from a set of patients (e.g., cancer patients; etc.); generating one or more sequencing libraries (e.g., suitable for generating sequencing outputs indicative of biomarkers associated with patient responsiveness to one or more therapies; etc.) based on processing of the biological samples; determining sets of sequencing reads (e.g., for cDNA sequences derived from cDNA converted from mRNA indicating expression levels for PD-L1 and CD8A; etc.) for the set of patients based on the one or more sequencing libraries; processing the sequencing reads for determining immune response-associated data (e.g., PD-L1 gene expression levels; CD8A gene expression levels; chimeric transcripts indicative of gene fusion; cDNA sequence data, such as from cDNA converted from mRNA; DNA sequence data; TMB-associated data; MSI-associated data; etc.); determining treatment response characterizations (e.g., associated with patient sensitivity to one or more immune checkpoint therapies such as PD-1/PD-L1 inhibitors; etc.) for the set of patients based on the immune response-associated data (e.g., based on independent and/or combined analyses of the different types of immune response-associated data; etc.); and facilitating treatment provision for one or more patients of the set of patients based on the treatment response characterizations (e.g., identifying a subset of patients with indications of positive responsiveness to therapies for clinical trials, such as for clinical trial enrollment; providing the treatment response characterizations to one or more care providers, such as for guiding care decisions by the one or more care providers; etc.).

Embodiments of the methods and systems disclosed herein (e.g., method 100 or a system 200) can function to enrich, identify, select, and/or otherwise characterize a patient population as responsive to one or more immune checkpoint therapies (e.g., PD-1/PD-L1 inhibitors) and/or other suitable therapies based on a plurality of different types of immune response-associated data, such as including two or more of PD-L1 gene expression levels, CD8A gene expression levels, chimeric transcripts indicative of gene fusion, cDNA sequence data (e.g., such as from cDNA converted from mRNA; etc.), DNA sequence data, TMB-associated data, MSI-associated data, and/or other suitable types of immune response-associated data.

In specific examples, data regarding predictive biomarkers (and/or other suitable immune response-associated data) can be analyzed in generating one or more treatment response characterizations for one or more patients, in order to predict patient benefit from checkpoint inhibitors, such as inhibitors that block PD-1/PD-L1 activity (e.g., thereby enabling a patient immune response to improve a cancer condition and/or other suitable conditions in the patient; etc.), such as where the different types of immune response-associated data can independently and/or in any suitable combination contribute to the predictiveness of patient response.

In specific examples, treatment response characterizations (e.g., indicating patient responsiveness to checkpoint inhibitor therapies, etc.) can be used for clinical trials (e.g., clinical trial enrollment and patient selection; stratification of patient populations, such as based on different combinations of biomarkers; therapy characterization; results analysis; and/or other suitable purposes related to clinical trials; etc.), care provision (e.g., providing treatment response characterizations to care providers for guiding care decisions regarding patients; therapy determination for patients; etc.), and/or other suitable applications. Additionally or alternatively, embodiments of the methods and systems disclosed herein (e.g., method 100 and/or system 200) can function to conserve valuable biological samples, such as lung cancer tissue biopsies, tumor specimens, and/or suitable types of biological samples. In specific examples, immune response-associated data collection can be performed based on RNA sequencing (e.g., sequencing of cDNA converted from mRNA, such as mRNA indicating expression of PD-L1 and/or CD8A; etc.) and/or other suitable processing approaches as an alternative to sample processing approaches that can require a relatively larger usage of biological sample (e.g., immunohistochemistry; etc.). However, embodiments of the methods and systems disclosed herein (e.g., method 100 and/or system 200) can include any suitable functionality.

Embodiments of the methods and systems disclosed herein (e.g., method 100 and/or system 200) can be performed for (e.g., in relation to evaluating gene expression levels; comparing against thresholds; determining treatment response characterizations; etc.) PD-L1 and/or CD8A exon junctions, including any one or more of: PD-L1 exons 3-4, PD-L1 exons 4-5, CD8A exons 4-5, and/or other suitable PD-L1 and/or CD8A exon junctions, and/or exon junctions for other suitable genes.

Embodiments of the methods and systems disclosed herein (e.g., method 100 and/or system 200) are preferably performed in relation to (e.g., for, regarding, about, associated with, etc.) patients with and/or otherwise associated with one or more cancer conditions (and/or other suitable immune response-associated conditions; etc.), including any one or more of: lung cancer, melanoma, kidney cancer, bladder cancer, breast cancer, esophagus cancer, colon cancer, biliary cancer, brain cancer, rectum cancer, endometrium cancer, lymphoma, ovary cancer, pancreas cancer, prostate cancer, sarcoma, stomach cancer, thyroid cancer, small intestine cancer, hepatobiliary tract cancer, urinary tract cancer, any cancer stage (e.g., stage III, stage IV, stage II, stage I, stage 0; etc.) and/or any suitable cancer conditions (e.g., pan cancer; etc.). Additionally or alternatively, immune response-associated conditions can include any one or more of: autoimmune disease; hepatitis; event-related immune response suppression (e.g., during tissue allografts, pregnancy, etc.).

Immune response-associated conditions can include any one or more of: symptoms, causes, diseases, disorders, associated risk, associated severity, and/or any other suitable aspects associated with immune response-associated conditions.

Embodiments of the methods disclosed herein preferably apply, include, and/or are otherwise associated with next-generation sequencing (NGS) (e.g., processing biological samples to generate sequence libraries for sequencing with next-generation sequencing systems; etc.). Embodiments of the methods disclosed herein can include, apply, and/or otherwise be associated with semiconductor-based sequencing technologies. Additionally or alternatively, embodiments of the methods disclosed herein can include, apply, and/or otherwise be associated with any suitable sequencing technologies (e.g., sequencing library preparation technologies; sequencing systems; sequencing output analysis technologies; etc.). Sequencing technologies preferably include next-generation sequencing technologies. Next-generation sequencing technologies can include any one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing and/or other suitable semiconductor-based sequencing technologies, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), sequencing-by-synthesis, tunneling currents sequencing, sequencing by hybridization, mass spectrometry sequencing, microscopy-based techniques, and/or any suitable next-generation sequencing technologies. In specific examples, embodiments of the methods disclosed herein can include applying next-generation sequencing technologies to sequence libraries prepared for facilitating generation of sequence reads associated with a plurality of biomarkers for responsiveness to one or more immune checkpoint therapies (e.g., PD-1/PD-L1 inhibitors; etc.).

Additionally or alternatively, sequencing technologies can include any one or more of: capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

Embodiments of the methods disclosed herein can include, apply, perform, and/or otherwise be associated with any one or more of: sequencing operations, alignment operation (e.g., sequencing read alignment; etc.), lysing operations, cutting operations, tagging operations (e.g., with barcodes; etc.), ligation operations, fragmentation operations, amplification operations (e.g., helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), etc.), purification operations, cleaning operations, suitable operations for sequencing library preparation, suitable operations for facilitating sequencing and/or downstream analysis, suitable sample processing operations, and/or any suitable sample- and/or sequence-related operations. In specific examples, sample processing operations can be performed for processing biological samples to generate sequencing libraries for facilitating characterization of a plurality of biomarkers associated with responsiveness to one or more immune checkpoint therapies.

Additionally or alternatively, data described herein (e.g., immune response-associated data, thresholds, models, parameters, normalized data, treatment response characterizations, treatment determinations, sample data, sequencing data, etc.) can be associated with any suitable temporal indicators (e.g., seconds, minutes, hours, days, weeks, time periods, time points, timestamps, etc.) including one or more: temporal indicators indicating when the data was collected, determined, transmitted, received, and/or otherwise processed; temporal indicators providing context to content described by the data; changes in temporal indicators (e.g., data over time; change in data; data patterns; data trends; data extrapolation and/or other prediction; etc.); and/or any other suitable indicators related to time. In specific examples, treatment response characterizations can be performed overtime for one or more patients, to facilitate patient monitoring, therapy effectiveness evaluation, additional treatment provision facilitation, and/or other suitable purposes.

Additionally or alternatively, parameters, metrics, inputs, outputs, and/or other suitable data can be associated with value types including any one or more of: binary values (e.g., binary status determinations of presence or absence of one or more biomarkers associated with positive responsiveness to immune checkpoint therapies and/or other suitable therapies, etc.), scores (e.g., aggregate scores indicative of a probability and/or degree of responsiveness to therapies described herein; etc.), values indicative of presence of, absence of, degree of responsiveness to one or more therapies described herein, classifications (e.g., patient classifications for sensitivity to therapies described herein; patent classifications based on absence or presence of different biomarkers of a set of biomarkers associated with responsiveness to therapies described herein, etc.), identifiers (e.g., sample identifiers; sample labels indicating association with different cancer conditions; patient identifiers; biomarker identifiers; etc.), values along a spectrum, and/or any other suitable types of values. Any suitable types of data described herein can be used as inputs (e.g., for different models; for comparison against thresholds; for portions of embodiments the method 100; etc.), generated as outputs (e.g., of different models; for use in treatment response characterizations; etc.), and/or manipulated in any suitable manner for any suitable components associated with embodiments of the methods disclosed herein.

One or more instances and/or portions of embodiments of the methods disclosed herein can be performed asynchronously (e.g., sequentially), concurrently (e.g., in parallel; concurrently on different threads for parallel computing to improve system processing ability for immune response-associated data processing and/or treatment response characterization generation; multiplex sample processing; multiplex sequencing such as for a plurality of biomarkers in combination, such as in a minimized number of sequencing runs; etc.), in temporal relation to a trigger event (e.g., performance of a portion of a method disclosed herein), and/or in any other suitable order at any suitable time and frequency by and/or using one or more instances of embodiments of inventions described herein.

Embodiments of a system (e.g., system 200) to perform the methods described herein can include one or more: sample handling systems (e.g., for processing samples; for sequencing library generation; etc.); sequencing systems (e.g., for sequencing one or more sequencing libraries; etc.); computing systems (e.g., for sequencing output analysis; for immune response-associated data collection and/or processing; for treatment response characterization generation; for any suitable computational processes; etc.); treatment systems (e.g., for providing treatment recommendations; for facilitating patient selection for clinical trials; for therapy provision; etc.); and/or any other suitable components.

Embodiments of the system and/or portions of embodiments of the system described herein can entirely or partially be executed by, hosted on, communicate with, and/or otherwise include one or more: remote computing systems (e.g., a server, at least one networked computing system, stateless, stateful; etc.), local computing systems, user devices (e.g., mobile phone device, other mobile device, personal computing device, tablet, wearable, head-mounted wearable computing device, wrist-mounted wearable computing device, etc.), databases (e.g., including sample data and/or analyses, sequencing data, user data, data described herein, etc.), application programming interfaces (APIs) (e.g., for accessing data described herein, etc.) and/or any suitable components. Communication by and/or between any components of the system and/or other suitable components can include wireless communication (e.g., WiFi, Bluetooth, radiofrequency, Zigbee, Z-wave, etc.), wired communication, and/or any other suitable types of communication.

Components of embodiments of methods and systems (e.g., system 200) described herein can be physically and/or logically integrated in any manner (e.g., with any suitable distributions of functionality across the components, such as in relation to portions of embodiments of the method 100; etc.). Portions of embodiments of methods and systems (e.g., system 200) described herein are preferably performed by a first party but can additionally or alternatively be performed by one or more third parties, users, and/or any suitable entities. However, of methods and systems (e.g., system 200) described herein can be configured in any suitable manner.

Embodiments of the methods disclosed herein (e.g., method 100) can include collecting immune response-associated data derived from one or more biological samples, which can function to collect (e.g., generate, determine, receive, etc.) data associated with immune response functionality, for enabling characterization of one or more patients in relation to responsiveness to one or more therapies described herein (e.g., PD-1/PD-L1 inhibitors; etc.) for one or more conditions described here (e.g., cancer conditions; etc.).

Immune response-associated data preferably includes data indicative of biological phenomena associated with (e.g., influencing, influenced by, related to, part of, including components of, etc.) the immune response and/or immune system; however, immune response-associated data can include any suitable data (e.g., derivable by sample processing techniques, bioinformatic techniques, statistical techniques, sensors, etc.) related to the immune response and/or immune system.

Types of immune response-associated data can include any one or more of: PD-L1 gene expression levels; CD8A gene expression levels; chimeric transcripts indicative of gene fusion; cDNA sequence data, such as from cDNA converted from mRNA; DNA sequence data; TMB-associated data; MSI-associated data; and/or any suitable types of immune response-associated data (e.g., for biomarkers associated with patient sensitivity to PD-1/PD-L1 inhibitors; etc.). Preferably, immune response-associated data includes a plurality of types, but any suitable number of types of immune response-associated data can be collected and/or used in generating one or more treatment response characterizations.

Collecting immune response-associates data preferably includes processing one or more biological samples for facilitating generation of the immune response-associated data. Biological samples preferably include tumor samples (e.g., tissue specimens, etc.) associated with one or more cancer conditions. In specific examples, biological samples can include formalin-fixed paraffin-embedded (FFPE) tumor specimens. In specific examples, FFPE tumor specimens can be used for isolation of mRNA (e.g., associated with gene expression of PD-L1 and gene expression of CD8A, etc.), which can be converted to cDNA and subsequently sequenced with a next-generation sequencing system (e.g., for determining gene expression levels; etc.) and/or suitable sequencing system. Additionally or alternatively FFPE tumor specimens and/or suitable biological samples can be used in preparing suitable sequencing libraries for subsequent sequencing and immune response-associated data collection associated with a plurality of biomarkers described herein in relation to responsiveness to immune checkpoint therapies such as PD-1/PD-L1 inhibitors. Biological samples can be derived from any suitable body region (e.g., a body region at which a cancer condition is present; etc.). Additionally or alternatively, biological samples can include any type of samples and/or number of samples for facilitating collection of immune response-associated data. Biological samples are preferably processed for facilitating characterization of a plurality of targets (e.g., corresponding to biomarkers associated with responsiveness to therapies described herein; etc.). In specific examples, sample processing can be performed for targeting specific loci (e.g., isolation and amplification of nucleic acids corresponding to the specific loci, such as through target-specific primers, etc.). Additionally or alternatively, sample processing can be performed for any suitable biological targets (e.g., associated with patient sensitivity to one or more immune checkpoint therapies such as PD-1/PD-L1 therapies; etc.). Biological targets (e.g., target markers; corresponding to, causing, contributing to, therapeutic in relation to, correlated with, and/or otherwise associated with one or more cancer conditions; targets of interest; known or identified targets; unknown or previously unidentified targets; etc.) can include any one or more of target sequence regions (e.g., sequence regions corresponding to biomarkers associated with patient sensitivity to PD-1/PD-L1 therapies; etc.), genes (e.g., PD-L1, CD8A, etc.), loci, peptides and/or proteins (e.g., antigens, immune cell receptors; antibodies etc.), carbohydrates, lipids, nucleic acids (e.g., messenger RNA, cDNA, DNA, microRNA, etc.), cells (e.g., whole cells, etc.), metabolites, natural products, and/or other suitable targets.

Any suitable number and type of biological samples from any suitable number and type of patients can be used in collecting immune response-associated data (e.g., sufficient immune response-associated data to be able to generate a sufficient treatment response characterization for facilitating treatment provision; etc.). In a specific example, a single biological sample can be processed and used for collecting (e.g., through processing of sequencing outputs; etc.): PD-L1 gene expression levels, CD8A gene expression levels, chimeric transcript data (e.g., indicating gene fusion, etc.), sequence variant data for cancer genes, TMB-associated data, and MSI-associated data. However, any suitable combination of such types of immune response-associated data can be collected from any suitable amount and type of biological samples.

Processing biological samples preferably includes performing sample processing operations (e.g., described herein, etc.) and next-generation sequencing (and/or other applying other suitable sequencing technologies described herein), but can additionally or alternatively include any suitable processing.

Sequencing outputs, any suitable data derived from biological samples and/or otherwise derived, immune response-associated data and/or other suitable data can be processed for determining immune response-associated data through applying, employing, performing, using, be based on, including, and/or otherwise being associated with one or more processing operations including any one or more of: sequence read quantification (e.g., sequence read processing and counting; etc.); sequence read identification (e.g., comparison to reference sequences; identifying sequence read correspondence to one or more biomarkers described herein; etc.); extracting features; performing pattern recognition on data, fusing data from multiple sources, combination of values, compression, conversion, performing statistical estimation on data (e.g., regression, etc.), wave modulation, normalization, updating, ranking, weighting, validating, filtering (e.g., for baseline correction, data cropping, etc.), noise reduction, smoothing, filling, aligning, model fitting, binning, windowing, clipping, transformations, mathematical operations (e.g., derivatives, moving averages, summing, subtracting, multiplying, dividing, etc.), data association, multiplexing, demultiplexing, interpolating, extrapolating, clustering, image processing techniques, other signal processing operations, other image processing operations, visualizing, and/or any other suitable processing operations.

In variations, collecting immune response-associated data can include collecting immune response-associated data from one or more subsets of patients (e.g., stratified patients, etc.), such as where subset determination can be based on presence, absence, and/or degree of different combinations of biomarkers (e.g., biomarkers described herein; etc.). In specific examples, collecting immune response-associated data can be performed for one or more studies evaluating therapy effectiveness for different subsets of patients stratified according to biomarker presence, absence, and/or degree. However, collecting immune response-associated data can be performed for any type and/or number of patients, and collecting immune response-associated data can be performed in any suitable manner.

Embodiments of the methods disclosed herein (e.g., method 100) can include determining a treatment response characterization associated with one or more therapies, based on the immune-response associated data, which can function to determine one or more characterizations indicative of responsiveness to one or more immune response-associated therapies, such as PD-1/PD-L1 inhibitors and/or other suitable immune checkpoint inhibitors (e.g., for use in evaluating potential treatment response; for use in otherwise facilitating treatment provision; etc.) and/or other suitable therapies described herein.

Treatment response characterizations preferably indicate the statuses for a plurality of biomarkers (e.g., biomarkers associated with patient sensitivity to therapies described herein; individual independent statuses for each biomarker of the plurality of biomarkers; a combined status for the plurality of biomarkers; etc.) but can additionally or alternatively indicate the status of a single biomarker. Treatment response characterizations can include one or more of: binary status indications (e.g., positive or negative for a given biomarker; present or absent for a given biomarker; etc.); values indicating degree (e.g., a score for a given biomarker indicating degree for that biomarkers, such as a degree of gene expression level for PD-L1 and/or CD8A; an aggregate score for overall responsiveness to one or more therapies described herein, such as calculated based on data for a plurality of biomarkers; etc.); probabilities (e.g., indicating risk associated with therapy provision; etc.); classifications (e.g., responsive or unresponsive classifications for a patient in relation to responsiveness to PD-1/PD-L1 inhibitors and/or suitable therapies described herein; etc.); recommendations (e.g., recommendations regarding specific therapies for different patients; etc.); labels (e.g., for stratifying patients; etc.); model outputs; processed immune response-associated data; raw immune response-associated data; information regarding immune response-associated conditions, therapies, biomarkers, and/or other suitable aspects; and/or other suitable types of data characterizing immune response in the context of patient conditions (e.g., cancer conditions, etc.) and therapy (e.g., immune checkpoint inhibitors; etc.).

In a specific example, a treatment response characterization can include simultaneous indications of PD-L1 and CD8A over-expression, TMB and MSI metrics (e.g., complementing PD-L1 and CD8A expression level data; etc.), mutations and gene fusions (e.g., relevant for therapy selection and/or evaluating PD-1/PD-L1 inhibitor therapy in the context of other potential therapies, etc.). Additionally or alternatively, treatment response characterizations can include indications for any suitable combination of biomarkers associated with any suitable number and/or type of therapies. However, treatment response characterizations can characterize any suitable aspects associated with the immune response and/or immune system, and/or can be configured in any suitable manner.

Determining one or more treatment response characterizations is preferably based on immune response-associated data. In examples, determining treatment response characterizations indicative of PD-L1 and/or CD8A can include identifying a patient as positive or negative for the respective biomarker (e.g., for PD-L1, for CD8A, etc.) based on comparing PD-L1 and CD8A expression levels (e.g., immune response-associated data collected from sequencing cDNA converted from mRNA corresponding to PD-L1 and CD8A) to respective thresholds (e.g., calling a patient positive for the biomarker in response to exceeding the threshold for the biomarker, and calling a patient negative for the biomarker in response to levels being below the threshold; etc.). In examples, determining treatment response characterizations indicative of gene fusion (e.g., which can facilitate a characterization indicating potential targeting by a therapy, such as EML4-ALK targetable by crizotinib; etc.) can include sequencing and/or otherwise analyzing chimeric transcripts (e.g., chimeric RNA, etc.). In examples, determining treatment response characterizations indicative of cancer gene sequence variants (e.g., which can indicate responsiveness to different therapies, such as EGFR mutations targetable by osimertinib, BRAF mutations targetable by vemurafenib, etc.) can include sequencing corresponding DNA (e.g., from a same biological sample used in collecting immune response-associated data of different types; etc.). In examples, determining treatment response characterizations indicative of TMB (e.g., which can be predictive of response to immune checkpoint inhibitors; etc.) can include counting the number of observed somatic mutations per megabase. In examples, determining treatment response characterizations indicative of MSI can include analyzing sequencing data (e.g., sequence reads, sequencing outputs, etc.) corresponding to microsatellite regions (e.g., loci corresponding to MSI; etc.). Generating treatment response characterizations indicative of a plurality of biomarkers (e.g., described herein) can improve the characterization of patient responsiveness to PD-1/PD-L1 inhibitor therapy and/or other suitable therapies described herein, such as for improved facilitation of treatment provision for one or more conditions described herein.

Additionally or alternatively, determining one or more treatment response characterizations, determining one or more treatment response characterization models, suitable portions of embodiments of the methods described herein (e.g., method 100), and/or suitable portions of embodiments of the systems described herein (e.g., system 200), can include, apply, employ, perform, use, be based on, and/or otherwise be associated with one or more processing operations including any one or more of: processing immune response-associated data; extracting features (e.g., associated with responsiveness to one or more therapies described herein; etc.), performing pattern recognition on data, fusing data from multiple sources, combination of values (e.g., averaging values, etc.), compression, conversion, performing statistical estimation on data, wave modulation, normalization, updating, ranking, weighting, validating, filtering (e.g., for baseline correction, data cropping, etc.), noise reduction, smoothing, filling, aligning, model fitting, binning, windowing, clipping, transformations, mathematical operations (e.g., derivatives, moving averages, summing, subtracting, multiplying, dividing, etc.), data association, multiplexing, demultiplexing, interpolating, extrapolating, clustering, image processing techniques, other signal processing operations, other image processing operations, visualizing, and/or any other suitable processing operations.

Determining one or more treatment response characterizations can include performing one or more normalization processes, such as for enabling sequencing outputs (e.g., associated with any suitable biomarkers described herein, etc.) to be comparable to thresholds and/or across different sequencing runs. In examples, determining treatment response characterizations can include background-subtracting sequence read counts; and normalizing the background-subtracted sequence read counts into normalized reads per million (nRPM). In a specific example (e.g., for PD-L1 and/or CD8A), a fold-change ratio can be determined for a given gene (and/or suitable biomarker), according to: Ratio=Background Subtracted Read Count/Reads Per Million (RPM) profile. In a specific example, the RPM profile can be determined based on an average RPM (and/or other suitable aggregate RPM metric) of a plurality of replicates of biological samples across different validation sequencing runs. In a specific example, median values of determined ratios can be used for a Normalization Ratio for a given biological sample, where the nRPM can be calculated according to: nRPM=Background Subtracted Read Count/Normalization Ratio. Housekeeping genes usable for normalization processes (e.g., described herein) can include any one or more of: LRP1, MRPL13, TBP, HMBS, ITGB7, MYC, CIAO1, CTCF, EIF2B1, GGNBP2, SLC4A1AP and/or other suitable housekeeping genes (and/or any suitable genes). In some embodiments, two, three, four, five, six, seven, or eight of LRP1, MRPL13, TBP, HMBS, ITGB7, MYC, CIAO1, CTCF, EIF2B1, GGNBP2, SLC4A1AP are used for the normalization process. In some embodiments, three of LRP1, MRPL13, TBP, HMBS, ITGB7, MYC, CIAO1, CTCF, EIF2B1, GGNBP2, SLC4A1AP are used for the normalization process. In some embodiments, EIF2B1, HMBS, and CIAO1 are used for the normalization process. Additionally or alternatively, any suitable backgrounding and/or normalizing processes can be performed (e.g., for comparison of values to thresholds; for comparison of values across sequencing runs; etc.).

As noted above, determining one or more treatment response characterizations can be based on one or more thresholds (e.g., gene expression level thresholds). In variations, the methods disclosed herein (e.g., method 100) can include optimizing thresholds for comparisons to immune response-associated data and/or other suitable data for determining one or more indications of a treatment response characterization. In specific examples, determining thresholds can include: collecting samples from a set of patients with known response status; processing the samples to generate immune response-associated data; and processing the immune response-associated data along with treatment response data to derive appropriate thresholds corresponding to different biomarkers (e.g., PD-L1 gene expression level; CD8A gene expression level; etc.). In specific examples, normalized immune response-associated data (e.g., normalized sequencing data for PD-L1 gene expression data and CD8A gene expression data; etc.) can be compared against thresholds (e.g., where satisfying the threshold indicates a positive reading for the given biomarker; where failing the threshold indicates a negative reading for the given biomarker; etc.).

Determining one or more treatment response characterizations can include generating (e.g., training, etc.), applying, executing, updating, and/or otherwise processing one or more treatment response models, such as based on and/or using any suitable processing operations, artificial intelligence approaches, and/or suitable approaches described herein. Treatment response models can include any suitable number and type of weights, such as for applying different weights to different types of immune response-associated data and/or indications derived from the immune response-associated data (e.g., weighing PD-L1 and CD8A indications heavier than other types of biomarkers, in relation to determining responsiveness, such as in a form of a generalized response score, to PD-1/PD-L1 inhibitor therapy and/or other suitable therapies described herein; etc.).

Additionally or alternatively, determining treatment response models, treatment response models themselves, other suitable models (e.g., therapy recommendations models; etc.), suitable portions of embodiments of the method 100, suitable portions of embodiments of the system 200, can include, apply, employ, perform, use, be based on, and/or otherwise be associated with artificial intelligence approaches (e.g., machine learning approaches, etc.) including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach.

Treatment response models and/or any suitable models can include any one or more of: probabilistic properties, heuristic properties, deterministic properties, and/or any other suitable properties. Each model can be run or updated: once; at a predetermined frequency; every time a portion of an embodiment of the method 100 is performed; every time a trigger condition is satisfied (e.g., threshold updates; additional collection of biological samples and/or immune response-associated data; etc.), and/or at any other suitable time and frequency. Models can be run or updated concurrently with one or more other models, serially, at varying frequencies, and/or at any other suitable time. Each model can be validated, verified, confirmed, reinforced, calibrated, or otherwise updated based on newly received, up-to-date data; historical data or be updated based on any other suitable data. However, any suitable number and/or types of models can be applied in any suitable manner based on any suitable criteria.

However, determining treatment response characterizations can be performed in any suitable manner.

Embodiments of the methods disclosed herein (e.g., method 100) can additionally or alternatively include facilitating treatment provision for one or more patients based on the treatment response characterization, which can function to facilitate treatment provision for one or more users in relation to one or more patient conditions (e.g., cancer conditions; etc.). Facilitating treatment provision can include facilitating clinical trials based on the one or more treatment response characterizations for one or more patients, such as identifying the subsets of patients (e.g., with positive indications of biomarkers described herein) with greatest likeliness of positive response to therapies described herein (e.g., PD-1/PD-L1 inhibitor therapy, etc.). In a specific example, treatment response characterizations can be used in a tumor type-agnostic biomarker-guided investigation for maximize the identification of responsive patient subsets, such as in relation to PD-1/PD-L1 inhibitor therapy. In some embodiments, the methods disclosed herein to determine whether a cancer is a checkpoint inhibitor responsive cancer are provided to a health professional for determination of whether to treat the cancer with a checkpoint inhibitor. In some embodiments, the methods disclosed herein to determine whether a cancer is a checkpoint inhibitor responsive cancer are used to inform a health care professional whether or not to teach a cancer with a checkpoint inhibitor.

Facilitating treatment provision can additionally or alternatively include any one or more of: transmitting and/or presenting treatment response characterizations (e.g., to any suitable entities, such as clinical trial administrators, care providers, etc.); guiding care decision-making, such as is in relation to experiment administration (e.g., clinical trial administration), healthcare, and/or other suitable processes; determining one or more therapies (e.g., using a treatment model; therapies described herein; etc.) for one or more conditions (e.g., described herein; etc.); providing recommendations regarding treatments, treatment responses, and/or other suitable aspects; and/or other suitable processes associated with treatment provision. Therapies can include any one or more of: cancer therapies (e.g., PD-1/PD-L1 inhibitors, other checkpoint inhibitors, pembrolizumab, durvalumab, avelumab, atezolizumab, nivolumab; other immunotherapy agents; any suitable immune therapy treatments; etc.); consumables; drugs; surgical procedures; any suitable treatments associated with one or more conditions; and/or any suitable treatments. However, facilitating treatment provision can be performed in any suitable manner.

Embodiments of the methods and systems disclosed herein (e.g., method 100 and/or system 200) can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the methods and systems (e.g., method 100 and/or system 200) can be embodied and/or implemented at least in part as a machine configured to receive a computer-readable medium storing computer-readable instructions. The instructions can be executed by computer-executable components that can be integrated with embodiments of the system 200. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the methods and systems disclosed herein (e.g., method 100, system 200), and/or variants without departing from the scope defined in the claims. Variants described herein not meant to be restrictive. Certain features included in the drawings may be exaggerated in size, and other features may be omitted for clarity and should not be restrictive. The figures are not necessarily to scale. Section titles herein are used for organizational convenience and are not meant to be restrictive. The description of any variant is not necessarily limited to any section of this specification.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); RobertS. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

The present disclosure utilizes a next-generation sequencing (NGS) based assay that uses targeted high throughput parallel-sequencing technology for the detection of mutations, small frame preserving insertions/deletions (indels), amplifications, deep deletions, de novo deleterious mutations, gene fusion events, microsatellite instability (MSI), tumor mutation burden/load (TMB/TML), and individual non-chimeric gene expression transcripts on a single NGS run. The StrataNGS test is a laboratory-developed test (LDT) performed in a Clinical Laboratory Improvement Amendments (CLIA) certified and College of American Pathologist (CAP) accredited laboratory and is intended to be performed with serial number-controlled instruments and qualified reagents. This test was designed to focus on identification of clinically actionable genetic variants for which there is an approved therapy or clinical trial with established proof of concept.

The StrataNGS test is a solid tumor, pan-cancer test that combines tumor mutation load (TML; also referred to as tumor mutation burden (TMB)) and gene expression (non-chimeric transcripts) assessment capabilities with all elements of the clinically validated StrataNGS gene panel. The test utilizes Ampliseq chemistry for library creation, followed by ThermoFisher Ion S5XL or S5 Prime sequencing workflow. The test runs multiple patient samples on one Ion 550 chip, utilizing both DNA and RNA from each sample.

Tumor mutation burden includes non-coding (at highly characterized genomic loci) and coding, synonymous and non-synonymous, single and multi-nucleotide (two bases) variants present at >10% variant allele frequency (VAF); mutation rate per megabase (Mb) estimate and associated 90% confidence interval are calculated via the total number of positions with sufficient depth of coverage necessary for definitive assessment (maximum possible 1.7 Mb). Qualitative TMB results (low: <10 mutations per Mb, intermediate: 10-15 mutations per Mb, high: 15+ mutations per Mb) are reported. PD-L1 expression (normalized to multiple housekeeping genes and a common control) is reported as RNA Expression Score (RES, range 0-100), which represents the % of maximum PD-L1 expression observed across StrataNGS tested tumor samples. For samples with at least 50% tumor content, a RES threshold of >20.3 to define PD-L1 RNA High; this threshold was validated as 100% sensitive and 70% specific for predicting PD-L1 tumor proportion score (TPS)≥50%. For samples with <50% tumor content, the RES is reported but qualified with the potential impact of non-tumor cells on the RES. Strata Immune Signature is a novel combination biomarker comprised of PD-L1 expression, CD8A expression, and tumor content (40% or higher tumor content is required for a Strata Immune Signature High result).

The StrataNGS LDT was developed and the performance characteristics determined through validation by Strata Oncology. Strata Oncology has validated the performance of the entire non-fusion gene expression panel used on the StrataNGS LDT through representative validation in comparison to quantitative reverse transcription PCR (qRT-PCR) orthogonal test results, including both CD274 (PD-L1) and CD8A.

Recently, pembrolizumab was approved for patients with MSI-H or deoxyribonucleic acid (DNA) mismatch repair defects, irrespective of tumor type (Le et al, 2017). The registration-enabling clinical trial was conducted as an investigator-initiated trial and enrolled biomarker-positive patients across a range of tumor types. Fifty-four percent (54%; 95% confidence interval 39% to 69%) of patients had an objective response at 20 weeks and 1-year overall survival estimate of 76% (Le et al, 2017). MSI-H is more common in colorectal (17%) and endometrial cancer (28%) but is relatively rare in other tumor types, ranging from 0.2% to 5.4% across 16 cancer types (Ashktorab et al, 2016; Cortes-Ciriano, et al, 2017). MSI-H is thought to confer sensitivity to checkpoint inhibitors due to the substantially increased tumor mutational burden in MSI-H tumors, leading to an abundance of neoantigens and a robust tumor immune response, which is abrogated through immune checkpoint pathways.

Although representing the first tumor-agnostic biomarker-based drug approval, MSI-H tumors are speculated to represent only a fraction of tumor types outside of approved indications that are likely to respond to checkpoint therapy. For example, cancer patients who are TMB-H, but negative for MSI-H, or with expression markers indicative of a “checked” tumor immune response (eg, PD-L1, cluster of differentiation 8A [CD8A], interferon gamma) may be more likely to respond to checkpoint inhibition, independent of tumor type.

The Strata Immune Signature biomarker subgroup was identified through prospective assessment of StrataNGS on a retrospectively collected cohort through collaboration with the University of Michigan. The retrospective cohort included 150 patients previously treated with an approved immunotherapy (PD-L1/PD-1 inhibitor). Responders were defined as patients receiving immunotherapy for >12 months without documented disease progression (n=52, 35%), and nonresponders were defined as those progressing before 6 months (n=53, 35%). Excluded from the analyses were intermediate responders who were defined as patients receiving immunotherapy for 6 to 12 months (n=45, 30%). Among the 105 responders and nonresponders, 68 tumor samples across 10 tumor types were successfully tested with StrataNGS (n=32 responders and 36 nonresponders). None of the tumors tested were MSI H.

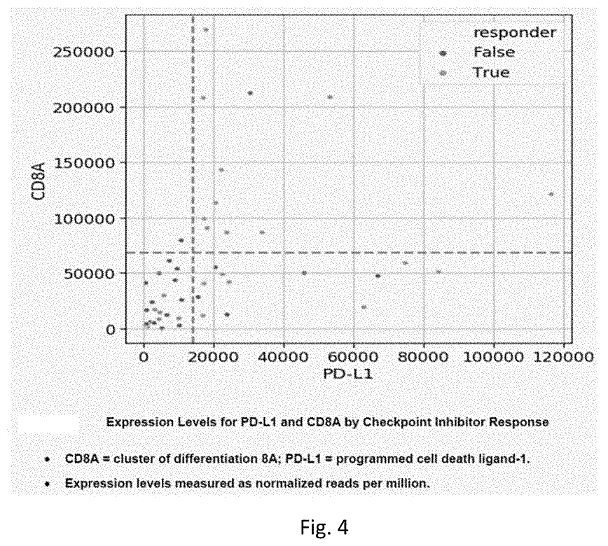

StrataNGS expression of 12 immunotherapy biomarkers were tested individually for association with checkpoint inhibitor response, and 5 genes (PD-L1, CD8A, IFNG, GZMA, and IDO1) were considered further (p<0.05). A random forest analysis was used to identify gene combinations that could more strongly enrich for response. Random forest analysis identified patients with combined PD-L1 high and CD8A high as enriched for responders. As shown in FIG. 4, initial thresholds were set by selecting the point on each biomarker's receiver-operating characteristic curve that maximized Youden's J statistic (14K normalized reads per million [nRPM] for PD-L1 and 69K nRPM for CD8A). Additionally, the PD-L1 threshold was independently verified by comparison with PD-L1 tumor proportion scores as determined by routine PD-L1 immunohistochemistry in an independent cohort of 80 samples. StrataNGS-defined PD-L1 high and CD8A high clearly separated a responder population in the context of samples with high tumor content (≥50%).

The Strata Immune Signature cohort (defined by PD-L1 high and CD8A high within samples containing ≥50% tumor content) included 10 responders and 1 nonresponder, the PD L1/CD8A low cohort included 7 responders and 5 nonresponders, and the PD-L1 low cohort included 6 responders and 17 nonresponders. Although the Strata Immune Signature is not a sensitive predictor of response, it is highly specific (as shown in FIG. 4), suggesting the potential for a high positive predictive value (ie, response rate) when used as a selection biomarker for checkpoint inhibitor therapy.

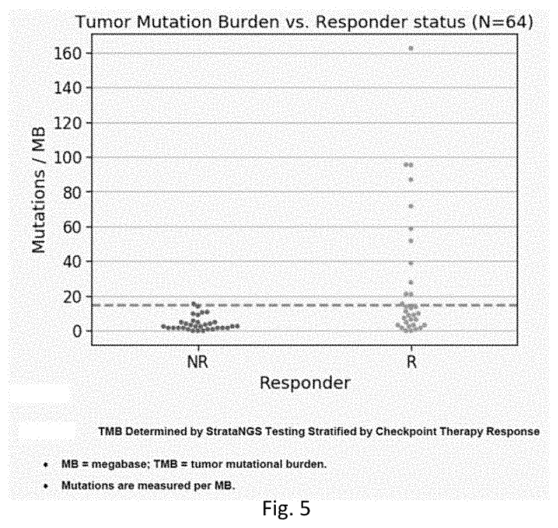

Sixty-four of the 80 samples in the independent cohort had sufficient material to also assess TMB by StrataNGS. Notably, all but one patient with TMB-H were responders (FIG. 5F; TMB H with 12 responders, 1 nonresponder).

Assuming a pan-cancer unselected checkpoint inhibitor response rate of 10%, adjusted positive predictive value (PPV) and negative predictive value were calculated. PD-L1 high demonstrated sensitivity of 70.8% and specificity of 72.7% but an adjusted PPV of 22.4%, whereas Strata Immune Signature has lower sensitivity (41.7%) but improved adjusted PPV (50.5%) and specificity (95.5%).

Similarly, TMB-H demonstrated less than 50% sensitivity but specificity of 100% and adjusted PPV of 100%. Sensitivity of an algorithm that included either Strata Immune Signature or TMB-H was >70% with an adjusted PPV of 63.4%. Assuming the observed characteristics, enrolling these 2 biomarker populations has the opportunity to capture 70% of all potential responders. The estimated frequency of the Strata Immune Signature is 6.4%, and TMB≥15 is 3.6% based on available data within the Strata Trial.

While it is estimated that TMB-H and Strata Immune Signature biomarkers exhibit a small degree of overlap (˜7.5%), they provide independent information and potential for predicting response to checkpoint inhibitors.

### Example 2—SIS Refinement

Final development work consisted of optimizing RNA expression dynamic range and quality control through both laboratory workflow and informatics refinements. Three primary changes were adopted:

1—The laboratory workflow was modified to adopt the assay manufacturer's recommendation of 20 cycles of PCR amplification for RNA quantification applications. This is in contrast to the 30-cycle amplification protocol originally employed. The change resulted in generally higher dynamic range and reduced coefficient of variation across technical replicates.

2—The set of housekeeping genes used for expression normalization was pruned from eight genes down to the three genes with the most stable expression values across all clinical and control replicate samples processed to date.

3—Confirmatory measurements are now considered when assessing Strata Immune Signature status. StrataNGS contains two independent amplicons for assessing PD-L1 expression levels; when the primary PD-L1 amplicon is above threshold, the result is qualified by ensuring the population percentile value of the secondary amplicon's measurement is greater than or equal to 80% of the primary amplicon's population percentile value. Similarly, above threshold measurements for CD8A are qualified by GZMA expression percentile at or above 80% of the CD8A percentile.

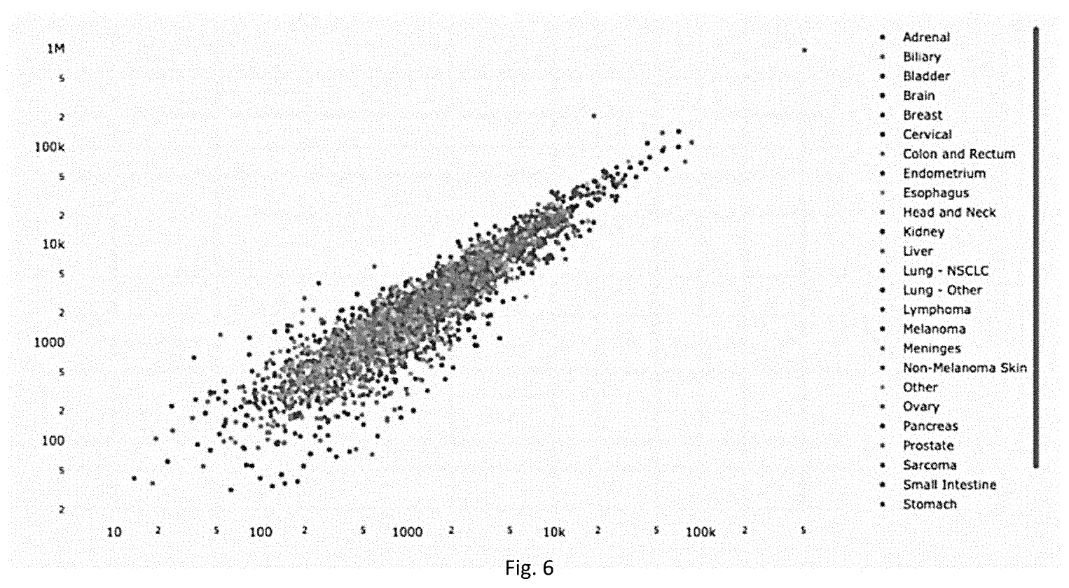

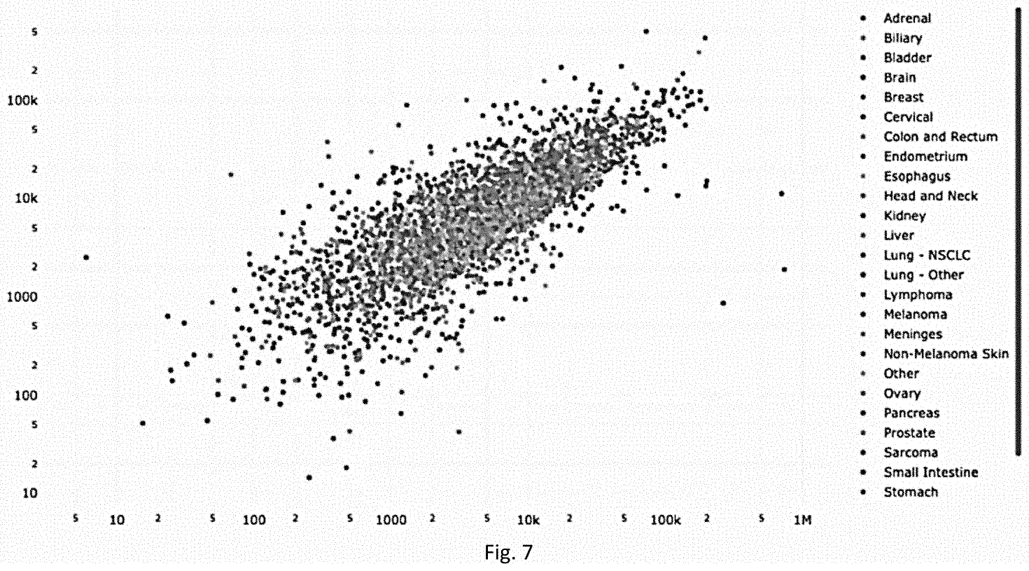

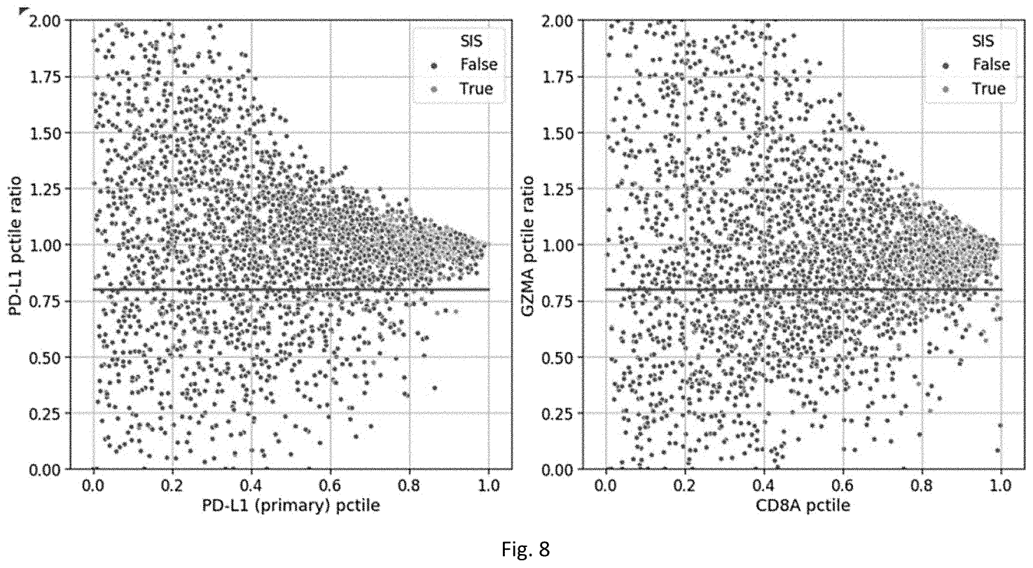

Concordance between the PD-L1 primary amplicon and secondary amplicon is shown in FIG. 6. Concordance between CD8A primary amplicon and GZMA amplicon is shown in FIG. 7. FIG. 8 provides graphs showing percentile ratios between PD-L1 amplicons (left side) or GZMA and CD8A (right side). SIS positive tumors (PD-L1 high, CD8A high, and tumor content 40% or more) are shown in orange. Approximately 2.2% of SIS positive tumors were disqualified by these confirmatory measurements (i.e., less than 0.8 ratio for PD-L1/PD-L1 or CD8A/GZMA), mostly due to low GZMA.

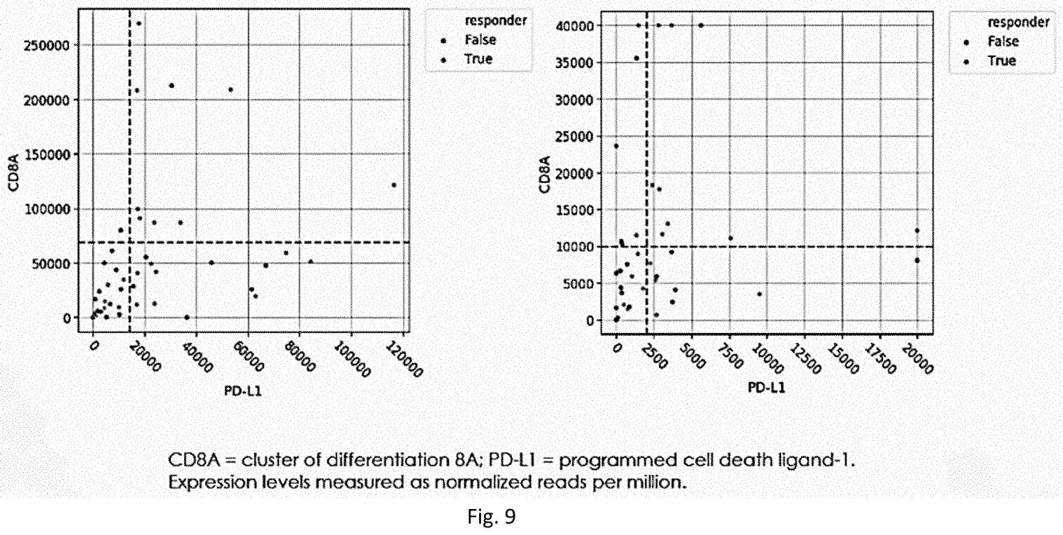

A comparison between the analysis in Example 1 and Example 2 is shown in FIG. 9.

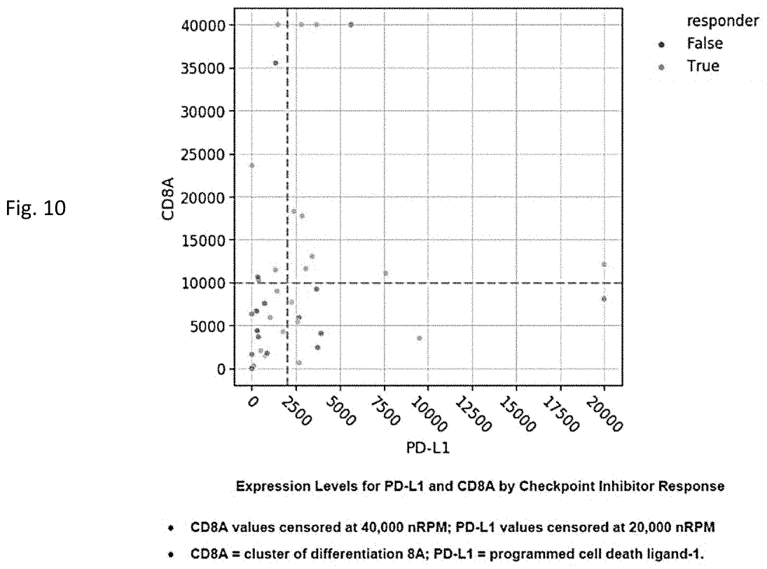

Refined Strata Immune Signature High is defined as: CD8A greater than or equal to 10,000 normalized reads per million (nRPM) (i.e., 67.6 percentile or more of CD8A expression in a population of tumor profiles) AND PDL1 greater than or equal to 2,000 nRPM (73.3 percentile or more of PD-L1 expression in a population of tumor profiles) AND Tumor Content greater than or equal to 40% AND secondary PDL1 measurement's percentile value is greater than or equal to 0.8*primary PDL1 measurement's percentile value AND GZMA percentile value is greater than or equal to 0.8*CD8A percentile value. After the refinement of the Strata Immune Signature High definition, the SIS cohort (defined by PD-L1 high and CD8A high within samples containing ≥40% tumor content) included 8 responders and 1 nonresponder, the PD L1/CD8A low cohort included 8 responders and 13 nonresponders, and the PD-L1 low cohort included 11 responders and 16 nonresponders. Although the Strata Immune Signature is not a sensitive predictor of response, it is highly specific (as shown in FIG. 10), suggesting the potential for a high positive predictive value (ie, response rate) when used as a selection biomarker for checkpoint inhibitor therapy.

Assuming a pan-cancer unselected checkpoint inhibitor response rate of 10%, adjusted positive predictive value (PPV) and negative predictive value were calculated. PD-L1 high demonstrated sensitivity of 54.2% and specificity of 72.7% but an adjusted PPV of 18.1%, whereas Strata Immune Signature has lower sensitivity (33.3%) but improved adjusted PPV (44.9%) and specificity (95.5%).

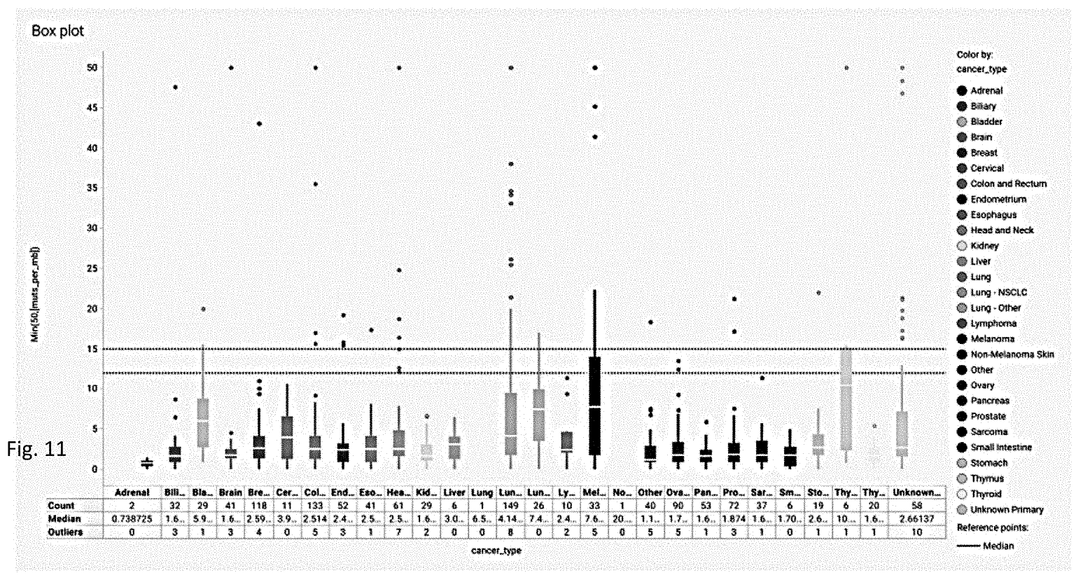

Similarly, a TMB-H screen (FIG. 11) demonstrated less than 50% sensitivity but specificity of 95.5% and adjusted PPV of 52.8%. The required tumor content for this screen is greater than or equal to 20%. TMB-H is defined as greater than 15 mutations per megabase.

Sensitivity of an algorithm that included either Strata Immune Signature or TMB-H was 66.7% with an adjusted PPV of 44.9%. Assuming the observed characteristics, enrolling these 2 biomarker populations has the opportunity to capture nearly 70% of all potential responders. The estimated frequency of the Strata Immune Signature is 7.6%, and TMB≥15 is 4.6% in the Strata Trial population.

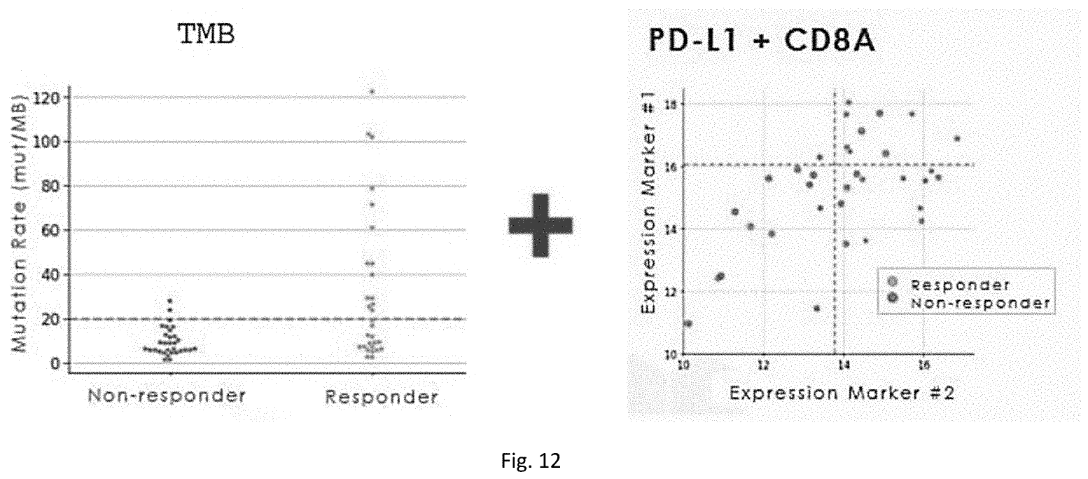

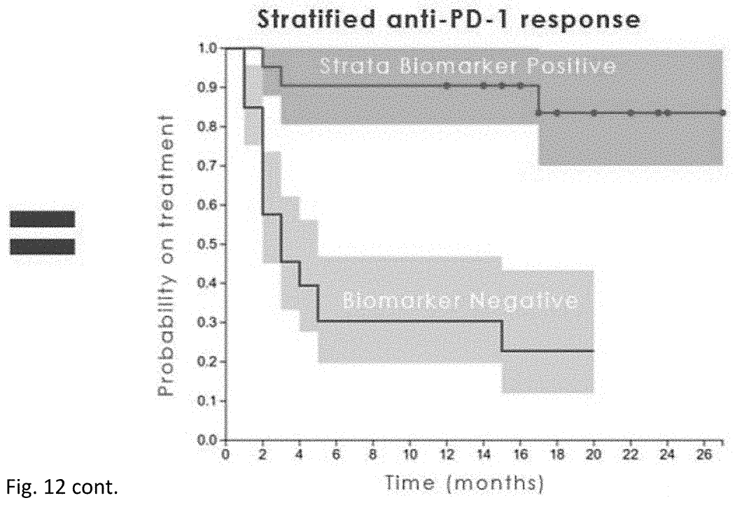

While it is estimated that TMB-H and Strata Immune Signature biomarkers exhibit a small degree of overlap (˜9.7%), they provide independent information and potential for predicting response to checkpoint inhibitors. Results for SIS positive or TMB positive patients are shown in FIG. 12 for tumors having a positive response to anti-PD-1 therapy.

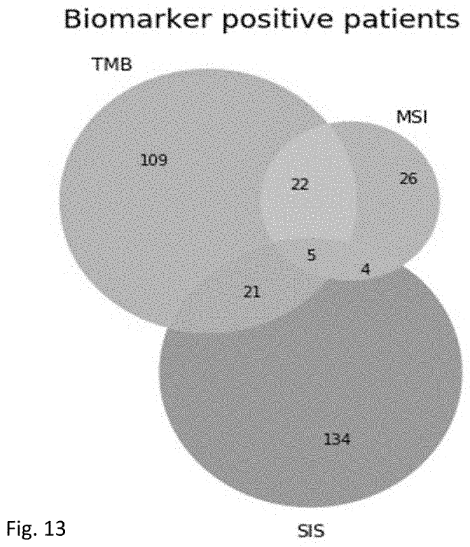

Comparison of TMB positive patients, MSI positive patients, and SIS positive patients is shown in FIG. 13. As is apparent, the SIS gene signature and TMB as claimed provide a different population of patients than MSI with checkpoint inhibitor responsive tumors and therefore provide a useful diagnostic tool for evaluating whether a subject should be administered a checkpoint inhibitor.

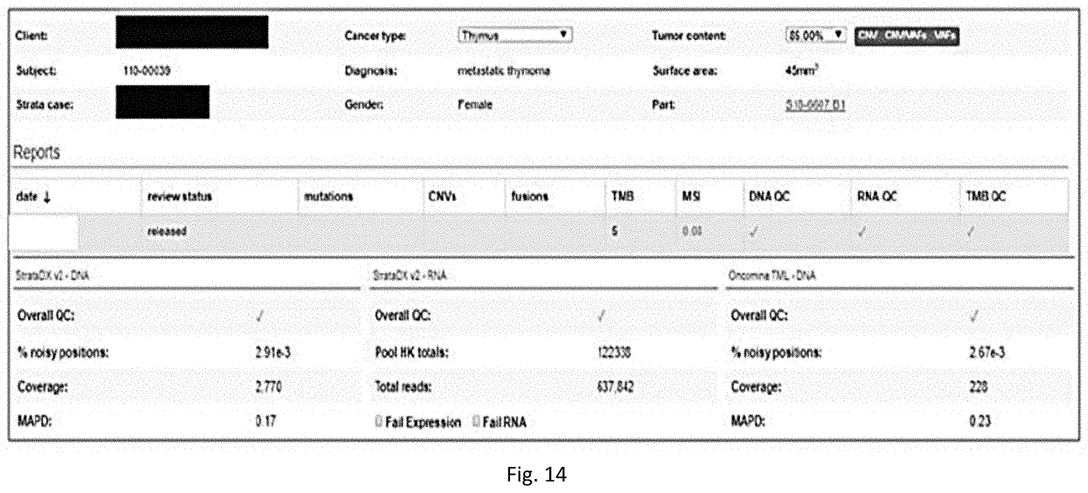

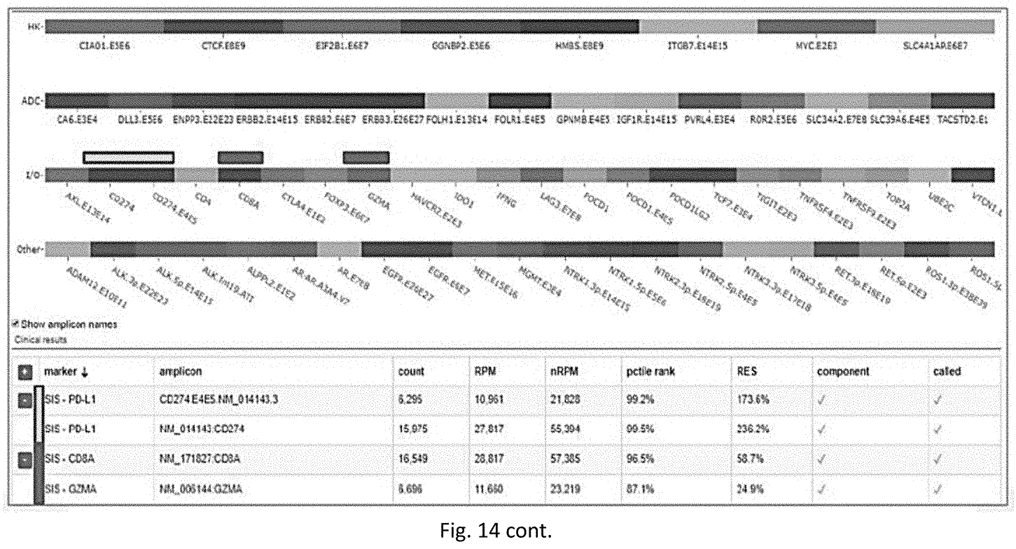

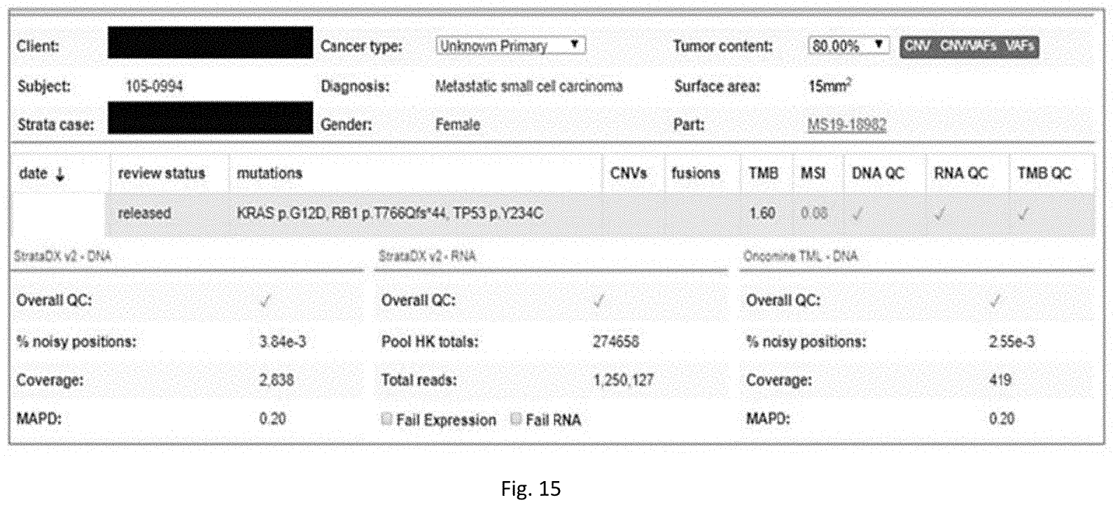

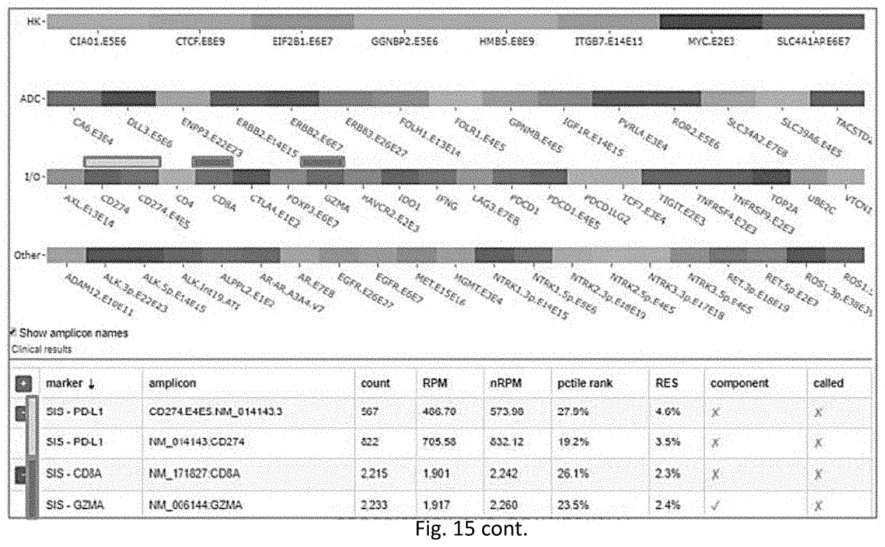

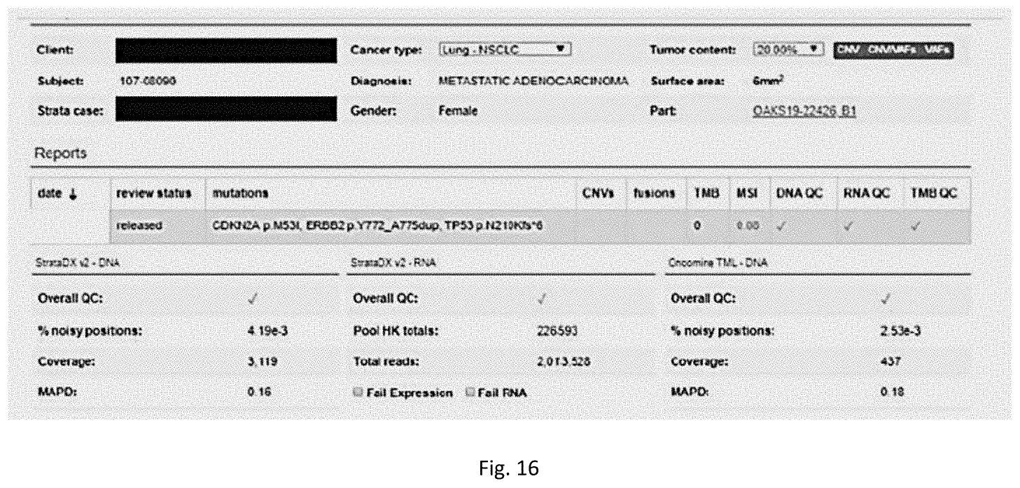

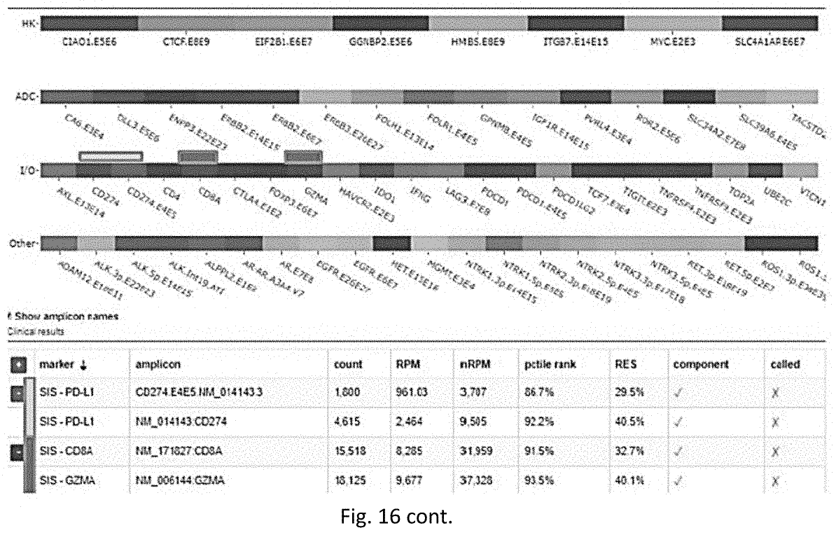

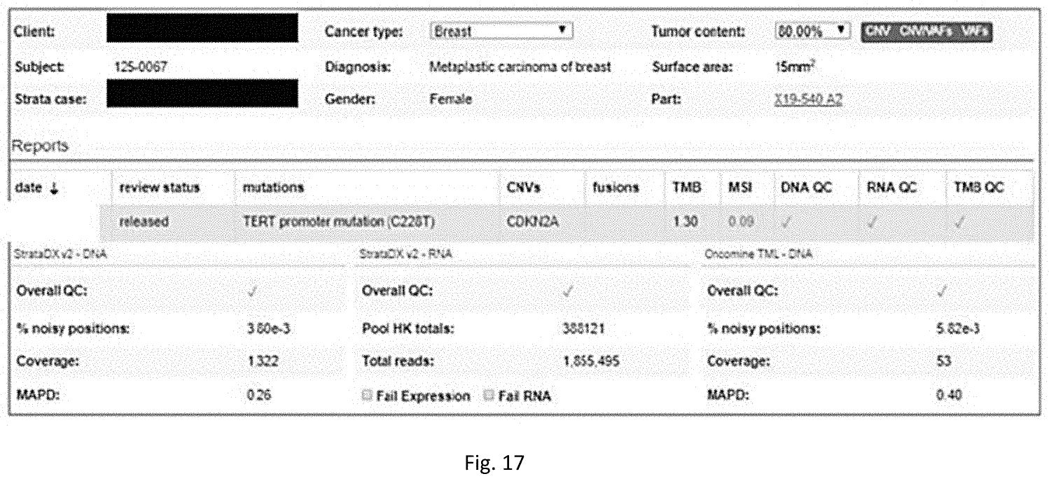

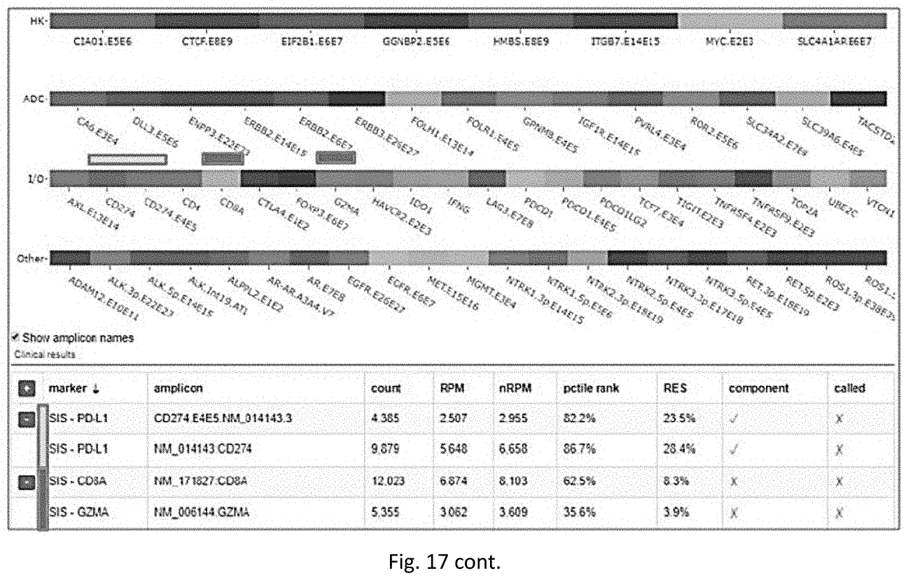

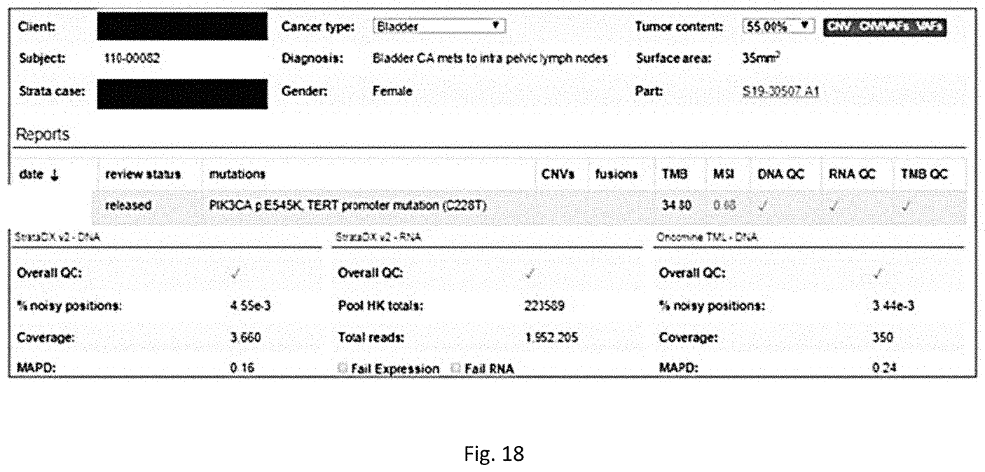

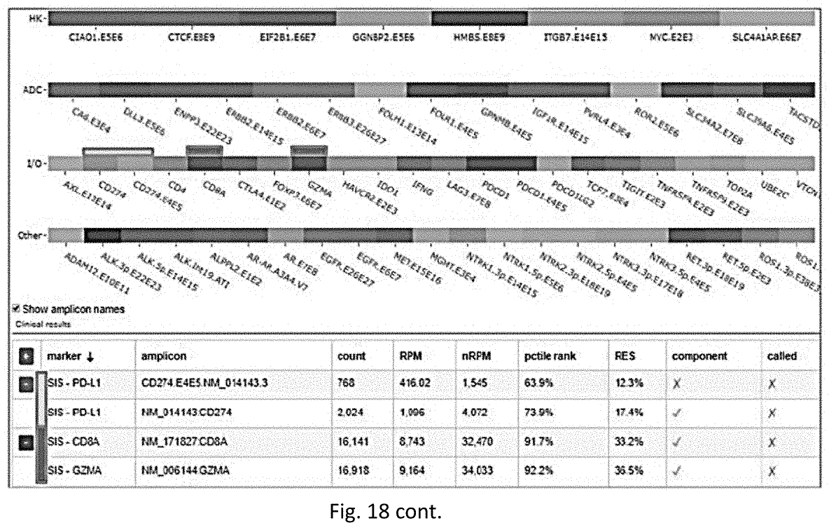

Example Scenarios for SIS screen are shown in FIGS. 14-18: PD-L1 High/CD8A High/TC High=SIS+(FIG. 14); PD-L1 Low/CD8A Low/TC High=SIS—(FIG. 15); PD-L1 High/CD8A High/TC Low=SIS—(FIG. 16); PD-L1 High/CD8A Low/TC High=SIS—(FIG. 17); PD-L1 Low/CD8A High/TC High=SIS—(FIG. 18).

1. A method of treatment comprising calculating PD-L1 expression, CD8A expression, and tumor content in a tumor specimen from a subject to identify the subject as having a checkpoint inhibitor responsive cancer; and administering a checkpoint inhibitor therapy to the identified subject.

2. The method of claim 1, wherein one or more of the following are also calculated for the tumor specimen: the presence of chimeric transcripts indicative of gene fusion, cDNA sequence data from cDNA converted from mRNA, DNA sequence data, tumor mutation burden (TMB)-associated data, and microsatellite instability (MSI)-associated data.

3. The method of claim 2, wherein tumor mutation burden (TMB)-associated data is also calculated for the tumor specimen.

4. The method of claim 1, wherein the tumor specimen is a formalin-fixed paraffin-embedded (FFPE) tumor specimen.

5. (canceled)

6. (canceled)

7. The method of claim 1, wherein the subject is identified as having a checkpoint inhibitor responsive cancer when the PD-L1 expression is calculated as high, wherein high PD-L1 expression equals at least the 73.3 percentile or more of PD-L1 expression in a population of tumor profiles.

8. (canceled)

9. The method of claim 7, wherein the calculated PD-L1 expression is confirmed with a secondary measurement of PD-L1 expression using a second amplicon, and wherein the secondary measurement's percentile value is 80% or more of the calculated PD-L1 expression value.

10. (canceled)

11. The method of claim 1, wherein the subject is identified as having a checkpoint inhibitor responsive cancer when the CD8A expression is calculated as high, wherein high CD8A expression equals at least the 67.6 percentile or more of CD8A expression in a population of tumor profiles.

12. (canceled)

13. The method of claim 11, wherein the calculated CD8A expression is confirmed with a secondary measurement of GZMA expression using a second amplicon, and wherein the secondary measurement's percentile value is 80% or more of the calculated CD8A expression value.

14. The method of claim 1, wherein the tumor specimen has a tumor content of 40% or more.

15. The method of claim 1, wherein the subject is identified as having a checkpoint inhibitor responsive cancer when the PD-L1 expression is calculated as high, the CD8A expression is calculated as high, and the tumor content of the tumor specimen is 40% or more.

16. (canceled)

17. The method of claim 1, wherein the checkpoint inhibitor is an anti-PD-1 antibody, an anti-CTLA-4 antibody, an anti-PD-L1 antibody, or an anti-PD-L2.

18. (canceled)

19. (canceled)

20. (canceled)

21. A method of identifying whether a subject has a checkpoint inhibitor responsive cancer comprising calculating PD-L1 expression, CD8A expression, and tumor content in a tumor specimen from a subject to identify whether the subject has a checkpoint inhibitor responsive cancer.

22. The method of claim 21, wherein one or more of the following are also calculated for the tumor specimen: the presence of chimeric transcripts indicative of gene fusion, cDNA sequence data from cDNA converted from mRNA, DNA sequence data, tumor mutation burden (TMB)-associated data, and microsatellite instability (MSI)-associated data.

23. The method of claim 22, wherein tumor mutation burden (TMB)-associated data is also calculated for the tumor specimen.

24. The method of claim 21, wherein the tumor specimen is a formalin-fixed paraffin-embedded (FFPE) tumor specimen.

25. (canceled)

26. (canceled)

27. The method of claim 21, wherein the subject is identified as having a checkpoint inhibitor responsive cancer when the PD-L1 expression is calculated as high, wherein high PD-L1 expression equals at least the 73.3 percentile or more of PD-L1 expression in a population of tumor profiles.

28. (canceled)

29. The method of claim 27, wherein the calculated PD-L1 expression is confirmed with a secondary measurement of PD-L1 expression using a second amplicon, and wherein the secondary measurement's percentile value is 80% or more of the calculated PD-L1 expression value.

30. (canceled)

31. The method of claim 21, wherein the subject is identified as having a checkpoint inhibitor responsive cancer when the CD8A expression is calculated as high, wherein high CD8A expression equals at least the 67.6 percentile or more of CD8A expression in a population of tumor profiles.

32. (canceled)

33. The method of claim 21, wherein the calculated CD8A expression is confirmed with a secondary measurement of GZMA expression using a second amplicon, and wherein the secondary measurement's percentile value is 80% or more of the calculated CD8A expression value.

34. The method of claim 21, wherein the tumor specimen has a tumor content of 40% or more.

35. The method of claim 21, wherein the subject is identified as having a checkpoint inhibitor responsive cancer when the PD-L1 expression is calculated as high, the CD8A expression is calculated as high, and the tumor content of the tumor specimen is 40% or more.

36. (canceled)
